(19) United States
(12) Patent Application Publication
(10) Pub. No.: US 2023/0025497 A1
(43) Pub. Date: Jan. 26, 2023

(54) DEVICES AND METHODS FOR MEASURING CARDIOGENIC AIRWAY MODULATION USING OPTICAL SENSING

(71) Applicant: Cardiac Pacemakers, Inc., St. Paul, MN (US)

(72) Inventors: Jeffrey E. Stahmann, Ramsey, MN (US); Keith R. Maile, New Brighton, MN (US)

(21) Appl. No.: 17/866,030

(22) Filed: Jul. 15, 2022

Related U.S. Application Data
(60) Provisional application No. 63/223,210, filed on Jul. 19, 2021.

(51) Int. Cl. A61B 5/11; A61B 5/00

(52) U.S. Cl. CPC A61B 5/1114; A61B 5/1126; A61B 5/6847; A61B 5/0059

(57) ABSTRACT

Embodiments herein relate to devices and methods for measuring cardiogenic airway modulations using optical sensing. In an embodiment, an optical cardiogenic modulation monitoring device can be included having an optical emitter configured to emit light at a first wavelength and an optical detector configured to detect incident light. The monitoring device can be configured so that light emitted from the optical emitter propagates through lung tissue. The monitoring device can also be configured to use detected incident light to measure cardiogenic oscillations of the lung tissue. Other embodiments are also included herein.

This application claims the benefit of U.S. Provisional Application No. 63/223,210, filed Jul. 19, 2021, the content of which is herein incorporated by reference in its entirety.

### FIELD

Embodiments herein relate to devices and methods for measuring cardiogenic airway modulations using optical sensing.

### BACKGROUND

Cardiogenic oscillations are heart-synchronous variations in the gas, flow, and pressure of the airway. It is widely accepted that cardiogenic oscillations are caused by blood flow and pressure changes due to the right ventricular contraction of the heart.

The magnitude and morphology of cardiogenic oscillations can provide insights into the status of a patient's respiratory system and, more broadly, a patient's health. For instance, a decrease in the magnitude of cardiogenic oscillations over time can indicate decreasing compliance of the respiratory system. Therefore, monitoring cardiogenic oscillations of a patient is an important factor to assess as part of evaluating the overall health of the patient as well as monitoring disease progression.

### SUMMARY

Embodiments herein relate to devices and methods for measuring cardiogenic airway modulations using optical sensing. In a first aspect, an optical cardiogenic modulation monitoring device can be included having an optical emitter configured to emit light at a first wavelength and an optical detector configured to detect incident light. The monitoring device can be configured so that light emitted from the optical emitter propagates through lung tissue. The monitoring device can also be configured to use detected incident light to measure cardiogenic oscillations of the lung tissue.

In a second aspect, in addition to one or more of the preceding or following aspects, or in the alternative to some aspects, the optical cardiogenic monitoring device can be configured to be implanted in a subcutaneous tissue of a patient.

In a third aspect, in addition to one or more of the preceding or following aspects, or in the alternative to some aspects, at least one of absorption, scattering and phase of the light detected by the optical detector can be used to measure cardiogenic oscillations of the lung tissue.

In a fourth aspect, in addition to one or more of the preceding or following aspects, or in the alternative to some aspects, the absorption can be used to determine an optical density of an optical path from the optical emitter to the optical detector and the optical density can be used to measure the cardiogenic oscillations of the lung tissue.

In a fifth aspect, in addition to one or more of the preceding or following aspects, or in the alternative to some aspects, both an AC component and an DC component of the light detected by the optical detector can be used to determine used to measure the cardiogenic oscillations of the lung tissue.

In a sixth aspect, in addition to one or more of the preceding or following aspects, or in the alternative to some aspects, the first wavelength can be from 800 nm to 1000 nm.

In a seventh aspect, in addition to one or more of the preceding or following aspects, or in the alternative to some aspects, the first wavelength can be from 150 nm to 250 nm.

In an eighth aspect, in addition to one or more of the preceding or following aspects, or in the alternative to some aspects, the device can include a second optical emitter, wherein the second optical emitter can be configured to emit light at a second wavelength.

In a ninth aspect, in addition to one or more of the preceding or following aspects, or in the alternative to some aspects, the second wavelength can be a near-infrared wavelength.

In a tenth aspect, in addition to one or more of the preceding or following aspects, or in the alternative to some aspects, the second wavelength can be from 750 nm to 1500 nm and different than the first wavelength.

In an eleventh aspect, in addition to one or more of the preceding or following aspects, or in the alternative to some aspects, the cardiogenic oscillations of the lung tissue can be measured based on a ratiometric calculation including the incident light on the detector originating from the light emitted at the first wavelength and the incident light on the detector originating from the light emitted at the second wavelength.

In a twelfth aspect, in addition to one or more of the preceding or following aspects, or in the alternative to some aspects, the device can include a housing can include a flexible member extending from the housing, wherein at least one of the optical emitter and the optical detector can be disposed on a flexible member.

In a thirteenth aspect, in addition to one or more of the preceding or following aspects, or in the alternative to some aspects, the optical emitter and the optical detector can be disposed on a substantially planar surface.

In a fourteenth aspect, in addition to one or more of the preceding or following aspects, or in the alternative to some aspects, the optical emitter can be disposed from 1 cm to 10 cm away from the optical detector.

In a fifteenth aspect, in addition to one or more of the preceding or following aspects, or in the alternative to some aspects, the monitoring device can be further configured to determine at least one of a magnitude and a morphology of the cardiogenic oscillations.

In a sixteenth aspect, in addition to one or more of the preceding or following aspects, or in the alternative to some aspects, the monitoring device can be configured to determine a trend in at least one of a magnitude and a morphology of the cardiogenic oscillations over a time period.

In a seventeenth aspect, in addition to one or more of the preceding or following aspects, or in the alternative to some aspects, the time period can be greater than one week.

In an eighteenth aspect, in addition to one or more of the preceding or following aspects, or in the alternative to some aspects, decreasing magnitude over time can be determined to reflect decreasing compliance of a respiratory system of a patient.

In a nineteenth aspect, in addition to one or more of the preceding or following aspects, or in the alternative to some aspects, the monitoring device can be configured to determine respiratory system mechanics based on the cardiogenic oscillations.

In a twentieth aspect, in addition to one or more of the preceding or following aspects, or in the alternative to some aspects, the optical cardiogenic monitoring device can be further configured to determine one of a heart rate, a respiratory rate, a tidal volume, or an extravascular lung water concentration.

In a twenty-first aspect, in addition to one or more of the preceding or following aspects, or in the alternative to some aspects, the light from the optical emitter propagates a depth of 1 cm to 5 cm into the lung tissue as measured from a surface of the optical cardiogenic monitoring device.

In a twenty-second aspect, in addition to one or more of the preceding or following aspects, or in the alternative to some aspects, further can include at least one of a pulse oximetry sensor, chemical sensor, posture sensor, and heart rate sensor.

In a twenty-third aspect, in addition to one or more of the preceding or following aspects, or in the alternative to some aspects, the cardiogenic oscillation measurements can be interpreted in view of at least one of heart rate, respiration, circadian rhythm, and posture.

In a twenty-fourth aspect, a method for measuring cardiogenic oscillations in a patient can be included, the method including implanting an optical cardiogenic monitoring device in a patient. The optical cardiogenic monitoring device can include an optical emitter and an optical detector. The method can include emitting light with the optical emitter into the lung tissue of the patient, detecting incident light with the optical detector, and measuring cardiogenic oscillations in the patient based on the light detected by the optical detector.

In a twenty-fifth aspect, in addition to one or more of the preceding or following aspects, or in the alternative to some aspects, wherein implanting the optical cardiogenic monitoring device includes implanting the optical cardiogenic monitoring device subcutaneously at or near a site of the lung tissue.

In a twenty-sixth aspect, in addition to one or more of the preceding or following aspects, or in the alternative to some aspects, wherein implanting the optical cardiogenic monitoring device subcutaneously includes implanting the optical cardiogenic monitoring device in an intercostal space at or near the site of the lung tissue.

In a twenty-seventh aspect, in addition to one or more of the preceding or following aspects, or in the alternative to some aspects, the optical cardiogenic monitoring device can be implanted at a distance of from 5 mm to 50 mm from a surface of the lung tissue.

In a twenty-eighth aspect, in addition to one or more of the preceding or following aspects, or in the alternative to some aspects, emitting light with the optical emitter into the lung tissue of the patient includes emitting the light at a wavelength from 800 nm to 1000 nm.

In a twenty-ninth aspect, in addition to one or more of the preceding or following aspects, or in the alternative to some aspects, emitting light with the optical emitter into the lung tissue of the patient includes emitting the light at least two distinct wavelengths.

In a thirtieth aspect, in addition to one or more of the preceding or following aspects, or in the alternative to some aspects, implanting an optical cardiogenic monitoring device in a patient includes orienting the optical cardiogenic monitoring device such that optical emitter and the optical detector face internally relative to a skin layer of the patient.

In a thirty-first aspect, in addition to one or more of the preceding or following aspects, or in the alternative to some aspects, emitting light with the optical emitter into the lung tissue of the patient includes propagating the light from 1 cm to 5 cm into the tissue of the patient relative to a surface of the optical cardiogenic monitoring device.

In a thirty-second aspect, an optical cardiogenic monitoring system can be included having a housing and an optical cardiogenic monitoring device. The optical cardiogenic monitoring device can include an optical emission assembly. The optical emission assembly can include a first optical emitter configured to emit light at a first wavelength, a second optical emitter configured to emit light at a second wavelength, and an optical detector configured to detect incident light. Light emitted from the optical emitter propagates through lung tissue and the detected incident light can be used to measure cardiogenic oscillations of the lung tissue. The optical cardiogenic monitoring device can be configured to be implanted in a subcutaneous tissue of a patient.

In a thirty-third aspect, in addition to one or more of the preceding or following aspects, or in the alternative to some aspects, at least one of absorption, scattering and phase of the light detected by the optical detector can be used to measure the cardiogenic oscillations.

In a thirty-fourth aspect, in addition to one or more of the preceding or following aspects, or in the alternative to some aspects, both an AC component and an DC component of the light detected by the optical detector can be used to measure the cardiogenic oscillations of the lung tissue.

In a thirty-fifth aspect, in addition to one or more of the preceding or following aspects, or in the alternative to some aspects, the first wavelength can be from 800 nm to 1000 nm.

In a thirty-sixth aspect, in addition to one or more of the preceding or following aspects, or in the alternative to some aspects, the first wavelength can be from 150 nm to 250 nm.

In a thirty-seventh aspect, in addition to one or more of the preceding or following aspects, or in the alternative to some aspects, the second wavelength can be a near-infrared wavelength.

In a thirty-eighth aspect, in addition to one or more of the preceding or following aspects, or in the alternative to some aspects, the second wavelength can be from 750 nm to 1500 nm.

In a thirty-ninth aspect, in addition to one or more of the preceding or following aspects, or in the alternative to some aspects, the cardiogenic oscillations of the lung tissue can be a function of the ratio of the detected light originating from the first optical emitter and the detected light originating from the second optical emitter.

In a fortieth aspect, in addition to one or more of the preceding or following aspects, or in the alternative to some aspects, the housing can include a flexible member extending from the housing, wherein at least one of the first optical emitter, the second optical emitter, and the optical detector can be disposed on a flexible member.

In a forty-first aspect, in addition to one or more of the preceding or following aspects, or in the alternative to some aspects, the optical emission assembly and the optical detector can be disposed on a substantially planar surface.

In a forty-second aspect, in addition to one or more of the preceding or following aspects, or in the alternative to some aspects, the optical emitter can be disposed from 1 cm to 10 cm away from the optical detector.

In a forty-third aspect, in addition to one or more of the preceding or following aspects, or in the alternative to some aspects, the optical cardiogenic monitoring device can be further configured to determine one of a heart rate, a respiratory rate, a tidal volume, or an extravascular lung water concentration.

In a forty-fourth aspect, in addition to one or more of the preceding or following aspects, or in the alternative to some aspects, the light from the optical emission assembly propagates a depth of 1 cm to 5 cm into the lung tissue as measured from a surface of the optical cardiogenic monitoring device.

In a forty-fifth aspect, in addition to one or more of the preceding or following aspects, or in the alternative to some aspects, the system further can include at least one of a pulse oximetry sensor, chemical sensor, posture sensor, and heart rate sensor.

In a forty-sixth aspect, in addition to one or more of the preceding or following aspects, or in the alternative to some aspects, the cardiogenic oscillations measurements can be interpreted in view of at least one of heart rate, respiration, circadian rhythm, and posture.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE FIGURES

Aspects may be more completely understood in connection with the following figures (FIGS.), in which:

FIG. 1 is a schematic view of an implanted optical cardiogenic modulation monitoring device in accordance with the embodiments herein.

FIG. 2 is a schematic view of an implanted optical cardiogenic modulation monitoring device system in accordance with the embodiments herein.

FIG. 3 is a schematic view of a wearable optical cardiogenic modulation monitoring device in accordance with the embodiments herein.

FIG. 4 is a schematic view of a wearable optical cardiogenic modulation monitoring device system in accordance with the embodiments herein.

FIG. 5 is a schematic view of a system including multiple implanted optical cardiogenic modulation monitoring devices in accordance with the embodiments herein.

FIG. 6 is a schematic top view of an optical cardiogenic modulation monitoring device in accordance with various embodiments herein.

FIG. 7 is a schematic top view of an optical cardiogenic modulation monitoring device in accordance with various embodiments herein.

FIG. 8 is a schematic top view of an optical cardiogenic modulation monitoring device in accordance with various embodiments herein.

FIG. 9 is a schematic top view of an optical cardiogenic modulation monitoring device in accordance with various embodiments herein.

FIG. 10 is a schematic cross-sectional view of an optical cardiogenic modulation monitoring device along line 10-10′ of FIG. 9 in accordance with various embodiments herein.

FIG. 11 is a schematic top view of an embodiment of an optical cardiogenic modulation monitoring device in accordance with various embodiments herein.

FIG. 12 is a schematic top view of an embodiment of an optical cardiogenic modulation monitoring device in accordance with various embodiments herein.

FIG. 13 is a schematic cross-sectional view of an implantable medical device in accordance with various embodiments herein.

FIG. 14 is a schematic top view of an embodiment of an optical cardiogenic modulation monitoring device in accordance with various embodiments herein.

FIG. 15 is a schematic top view of an embodiment of an optical cardiogenic modulation monitoring device in accordance with various embodiments herein.

FIG. 16 is a schematic top view of an embodiment of an optical cardiogenic modulation monitoring device in accordance with various embodiments herein.

FIG. 17 is a schematic cross-sectional view of a subcutaneous implantation site with an optical cardiogenic modulation monitoring device is shown in accordance with various embodiments herein.

FIG. 18 is a representative plot of water absorption of light at various wavelengths illustrating tissue penetration depth in accordance with various embodiments herein.

FIG. 19 is a schematic cross-sectional view of a human thorax with an implanted optical cardiogenic modulation monitoring device positioned therein to measure cardiogenic modulation in lung tissue in accordance with various embodiments herein.

FIG. 20 is a schematic cross-sectional view of a human thorax with a wearable optical cardiogenic modulation monitoring device positioned thereon to measure cardiogenic modulation in lung tissue in accordance with various embodiments herein.

FIG. 21 is a schematic representation of human lungs in accordance with various embodiments herein.

FIG. 22 is a diagram of various disease states impacting cardiogenic oscillations in accordance with various embodiments herein.

FIG. 23 is a representative plot of cardiogenic oscillations in accordance with various embodiments herein.

FIG. 24 is a schematic representation of an optical signal over time in accordance with various embodiments herein.

FIG. 25 is a schematic diagram of components of an optical cardiogenic modulation monitoring device in accordance with various embodiments herein.

FIG. 26 is a flow diagram of a method in accordance with various embodiments herein.

While embodiments are susceptible to various modifications and alternative forms, specifics thereof have been shown by way of example and drawings, and will be described in detail. It should be understood, however, that the scope herein is not limited to the particular aspects described. On the contrary, the intention is to cover modifications, equivalents, and alternatives falling within the spirit and scope herein.

### DETAILED DESCRIPTION

Embodiments herein relate to devices and methods for assessing cardiogenic oscillations using optical sensing. Various embodiments of devices and methods herein can be used for assessing cardiogenic oscillations status in tissues such as lung tissue. The systems, devices, and methods herein can be configured to determine and/or assess cardiogenic oscillations using optical sensing that can penetrate deeply into tissues, such as greater than one centimeter into tissues, such as pulmonary tissue.

Fluctuations in airway pressure and airway flow induced by cardiogenic oscillations can impact the optical path of light travelling through lung tissue and/or airway tissue. Changes to the optical path effect the properties of light received by a detector allowing for the magnitude and/or morphology of cardiogenic oscillations to be detected by optical cardiogenic modulation monitoring devices herein. Thus, in accordance with embodiments herein, cardiogenic oscillations can be assessed by measuring properties of light that propagates through the lung tissue and/or airway of a patient.

In accordance with embodiments herein light emitted from an optical emitter of an optical cardiogenic modulation monitoring device can propagate deeply into a patient's tissues and back to an optical detector of the optical cardiogenic modulation monitoring device. As light travels through the tissues of a patient, it can be affected by various optical phenomena including transmittance, reflectance, absorption, light attenuation, scatting, wavelength, current, and fluorescence. Devices and systems herein can be configured to utilize the optical properties of light having passed through deep tissue to sense physiological parameters such as cardiogenic oscillations at significant internal depths within a patient, such as where the depth is greater than one centimeter.

The optical cardiogenic modulation monitoring devices herein can be configured to cause light to penetrate deeply into the tissue by optimizing emitter-detector spacing, wavelength selection, and/or the use of multiple wavelengths.

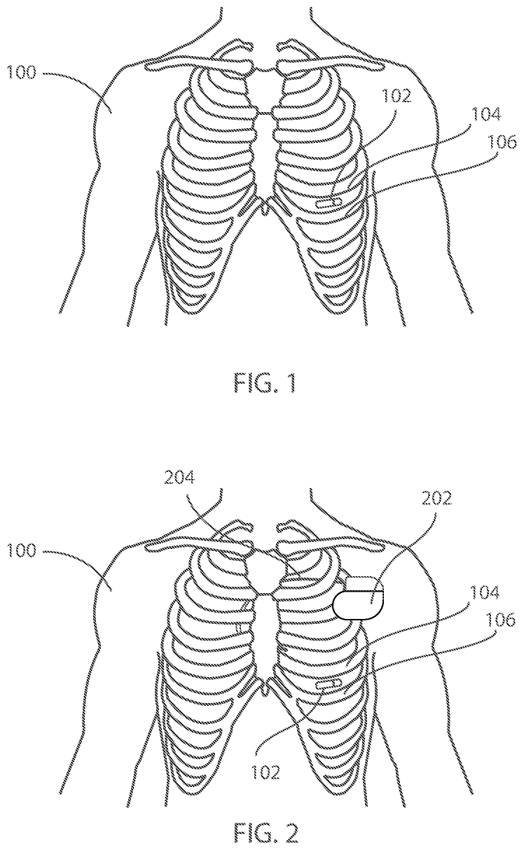

Devices and/or systems herein can take the form of implantable devices, wearable devices, or a combination of both. Specifically, devices and/or systems herein can include optical cardiogenic modulation monitoring devices that are implantable, wearable, or a combination of both. Referring now to FIG. 1, a schematic view of an implantable optical cardiogenic modulation monitoring device is shown in accordance with various embodiments herein. The optical cardiogenic modulation monitoring device 102 is shown in FIG. 1 as being positioned between the left fifth rib 104 and the left sixth rib 106 within the left fifth intercostal space of patient 100. While placement of the optical cardiogenic modulation monitoring device 102 is shown within the left fifth intercostal space, it will be appreciated that the optical cardiogenic modulation monitoring device 102 can be implanted within other locations of the body and, in particular, in or adjacent to other intercostal spaces along either the right side or the left side of a patient's rib cage or at other sites in the body including other anterior, lateral, posterior, superior or inferior thoracic locations.

The implantation depth of the optical cardiogenic modulation monitoring device can be from 1 millimeter (mm) to 25 mm or more below the skin layer of a patient's body. In some embodiments, the implantation depth below the skin layer can be greater than or equal to 1 mm, 2 mm, 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, 25 mm, 26 mm, 27 mm, 28 mm, 29 mm, or 30 mm, or can be an amount falling within a range between any of the foregoing.

In some embodiments, the implantation depth can be tailored to provide an optical cardiogenic modulation monitoring device to lung distance of from 1 mm to 45 mm or more. In some embodiments, the distance between the optical cardiogenic modulation monitoring device and lung can be greater than or equal to 1 mm, 2 mm, 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, 25 mm, 26 mm, 27 mm, 28 mm, 29 mm, 30 mm, 31 mm, 32 mm, 33 mm, 34 mm, 35 mm, 36 mm, 37 mm, 38 mm, 39 mm, 40 mm 41 mm, 42 mm, 43 mm, 44 mm, or 45 mm, or can be an amount falling within a range between any of the foregoing.

In some scenarios, the implantable optical cardiogenic modulation monitoring devices embodied herein can be used in combination with various types of implantable therapeutic or monitoring devices, including, but not limited to, implantable cardiac rhythm management devices, implantable pacemakers, implantable cardioverter-defibrillator devices, and the like. The implantable therapeutic devices can be implanted along with the optical cardiogenic modulation monitoring devices described herein. In some embodiments, optical cardiogenic modulation monitoring devices herein (or features and/or components thereof) can be integrated into implantable or wearable therapeutic devices.

Referring now to FIG. 2, a schematic view of an implantable optical cardiogenic modulation monitoring device is shown in accordance with various embodiments herein. The optical cardiogenic modulation monitoring device 102 is shown in FIG. 2 as being positioned between the left fifth rib 104 and the left sixth rib 106 within the left fifth intercostal space of patient 100. FIG. 2 further includes an implantable therapeutic device 202 positioned within the chest of the patient 100. Implantable therapeutic device 202 can be implanted at any position within patient 100 to provide a desired therapy to the patient in conjunction with signals determined by the optical cardiogenic modulation monitoring device, as will be discussed elsewhere herein. In some embodiments, implantable therapeutic device 202 can include one or more electrical stimulation leads 204 placed within the body of patient 100 at or near a treatment site.

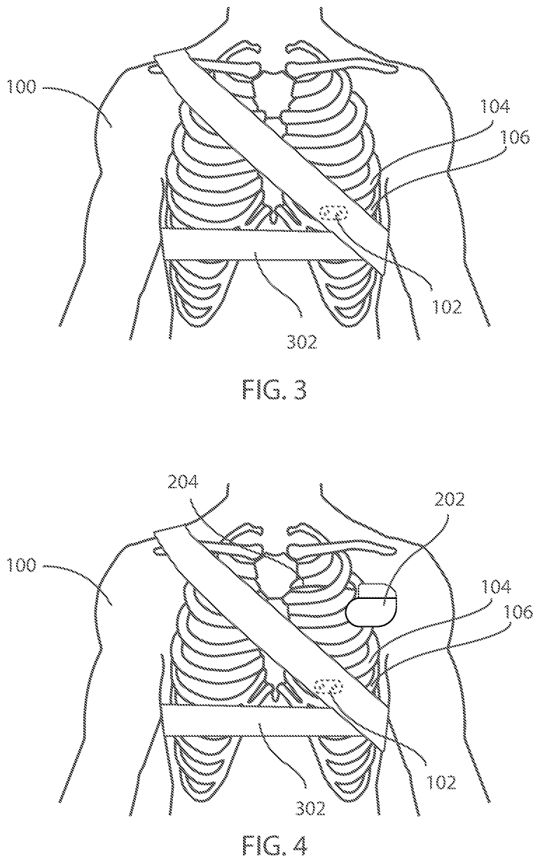

As referenced above, optical cardiogenic modulation monitoring devices herein can include those that are wearable devices. Referring now to FIG. 3, a schematic view of a wearable optical cardiogenic modulation monitoring device is shown in accordance with various embodiments herein. The optical cardiogenic modulation monitoring device 102 can be disposed along a length of a wearable strap 302 and externally positioned between the left fifth rib 104 and the left sixth rib 106 over the left fifth intercostal space of patient 100. While placement of the optical cardiogenic modulation monitoring device 102 is shown placed over the left fifth intercostal space, it will be appreciated that the optical cardiogenic modulation monitoring device 102 can also be positioned over or adjacent to other intercostal spaces along either the right side or the left side of a patient's rib cage. It will be appreciated that the wearable optical monitoring devices herein can be in direct contact with a patient's skin, or can be in direct contact with a protective barrier, gel, or film that is placed in direct contact with a patient's skin.

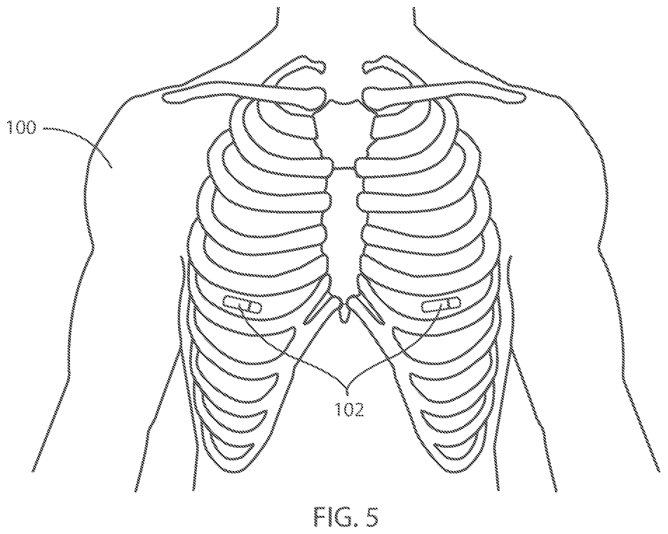

The wearable optical cardiogenic modulation monitoring devices embodied herein can be used in combination with various types of implantable therapeutic devices, such as those described above. The wearable oxygenation monitoring devices embodied herein can be used in combination with various types of implantable monitoring devices, such as implantable cardiac monitors. The implantable therapeutic or implantable monitoring devices can be implanted and used in a system in conjunction with the wearable optical cardiogenic modulation monitoring devices described herein. Referring now to FIG. 4, a schematic view of a wearable optical cardiogenic modulation monitoring device is shown in accordance with various embodiments herein. The wearable optical cardiogenic modulation monitoring device 102 can be disposed along a length of a wearable strap 302 and externally positioned between the left fifth rib 104 and the left sixth rib 106 over the left fifth intercostal space of patient 100 or at another site. FIG. 4 further includes an implantable therapeutic device 202 positioned within the chest of the patient 100. Implantable therapeutic device 202 can be implanted at any position within patient 100 to provide a desired therapy to the patient in conjunction with signals determined by the optical cardiogenic modulation monitoring device, as discussed elsewhere herein. Implantable therapeutic device 202 can include one or more electrical stimulation leads 204 placed within the body of patient 100 at or near a treatment site. In some embodiments, the wearable optical cardiogenic modulation monitoring devices herein can be or included with a patch sensor temporarily affixed to a patient by an adhesive, or it can be retained by a garment worn on the body of a patient. While not shown in the embodiments as shown in FIGS. 3 and 4, it will be appreciated that a combination of two or more optical monitoring devices can be worn on an exterior of a patient's body as components of the wearable straps 302.

In some embodiments only a single optical cardiogenic modulation monitoring device may be implanted while in other embodiments multiple optical cardiogenic modulation monitoring devices can be implanted within a patient. Referring now to FIG. 5, a schematic view of a system with multiple implantable optical cardiogenic modulation monitoring devices is shown in accordance with various embodiments herein. The optical cardiogenic modulation monitoring devices 102 are shown in FIG. 5 as being positioned between the fifth rib and the sixth rib within the left fifth intercostal space and right firth intercostal space, respectively. While placement of the optical cardiogenic modulation monitoring devices 102 are shown within the left fifth intercostal space and right fifth intercostal space, it will be appreciated that the optical cardiogenic modulation monitoring devices 102 can be placed within a combination of intercostal spaces along either the right side or the left side of a patient's rib cage or at other sites.

While FIG. 5 shows a combination of two optical cardiogenic modulation monitoring devices implanted within the intercostal spaces of patient 100, it will be appreciated that more than two optical cardiogenic modulation monitoring devices can be implanted within patient 100. In some embodiments, two, three, four, five, or more optical cardiogenic modulation monitoring devices can be implanted within patient 100. In some embodiments one or more of the oxygen monitoring devices is/are implanted and one or more of the oxygen monitoring devices is/are wearable.

The optical cardiogenic modulation monitoring devices herein can be configured to assess cardiogenic oscillations of a lung tissue. In various embodiments, the optical cardiogenic modulation monitoring devices herein can be further configured to determine a trend related to magnitude and/or morphology of the cardiogenic oscillations over a time period. In some embodiments, the optical cardiogenic modulation monitoring devices herein can be further configured to determine one or more of a heart rate, a respiratory rate, a tidal volume, or an extravascular lung water concentration, as discussed elsewhere herein. In some embodiments, the optical cardiogenic modulation monitoring devices herein can be further configured to determine respiratory system mechanics based on the cardiogenic oscillations.

In various embodiments, optical cardiogenic modulation monitoring devices can be implanted along with at least one secondary sensor. Secondary sensors can include, but are not to be limited to, a pulse oximetry sensor, a chemical sensor, a posture sensor, or a heart rate sensor. The secondary sensors can be used to determine one or more of a heart rate, a respiratory rate, a tidal volume, or an extravascular lung water concentration. In some embodiments, the secondary sensors can be integrated with the optical cardiogenic modulation monitoring device. In other embodiments, the secondary sensors can be physically separate from the optical cardiogenic modulation monitoring device, but in communication therewith.

It will be appreciated that the placement of the optical cardiogenic modulation monitoring device is not restricted to an intercostal space as depicted in FIGS. 1-5. Rather, the optical cardiogenic modulation monitoring device can be placed anywhere in the patient's body where light can propagate a sufficient depth into the body of a patient to measure cardiogenic oscillations at any location of the lung tissue and/or the airway (e.g., trachea, pharynx, nasal cavity, nostril, mouth).

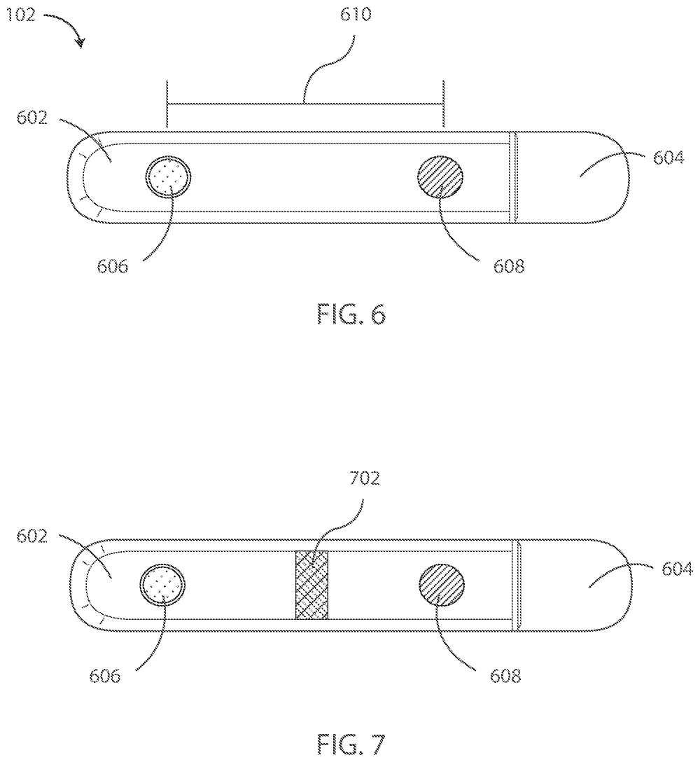

The optical cardiogenic modulation monitoring devices herein can include various components, such as optical emitters, optical detectors, secondary sensors, and optical barriers in various configurations in accordance with the embodiments herein and as shown and described in reference to FIGS. 6-13. It will be appreciated that in various embodiments, the optical emitters and optical detectors can be disposed together on the same side of the optical cardiogenic modulation monitoring device, while the secondary sensors can be disposed on the opposite side of the optical cardiogenic modulation monitoring device. In other embodiments, the optical emitters, optical detectors, and secondary sensors can be disposed together on the same side of the optical cardiogenic modulation monitoring device.

Referring now to FIG. 6, an optical cardiogenic modulation monitoring device 102 is shown in accordance with the embodiments herein. In this embodiment, the optical cardiogenic modulation monitoring device 102 can include a housing 602 and a header 604 coupled to the housing 602. Various materials can be used to form the housing 602 and the header 604. In some embodiments, the housing 602 can be formed of a material such as a metal, ceramic, a polymer, or a composite. In some embodiments, part of all of the housing 602 can be transparent to provide a window for optical components of cardiogenic modulation monitoring device 102. In some embodiments, the housing 602 can include a transparent window. The header 604 can be formed of various materials, and in some embodiments the header 604 can be formed of a translucent polymer such as an epoxy material. In some embodiments the header 604 can be hollow. In other embodiments the header 604 can be filled with components and/or structural materials such as epoxy or another material such that it is non-hollow. In yet other embodiments, the optical cardiogenic modulation monitoring device 102 can be devoid of a header 604 or can include a header at either end or both ends of the optical cardiogenic modulation monitoring device.

In various embodiments, the optical cardiogenic modulation monitoring device 102 can include a first optical emitter 606 and a first optical detector 608, each coupled to the housing 602. The first optical emitter 606 can be configured to emit light at a UV, visible, or near-infrared wavelength. The first optical emitter 606 can be configured to emit light at a first wavelength of from 100 nanometers (nm) to 2000 nm. In various embodiments, the first optical emitter can be configured to emit light at a first wavelength of from 600 nm to 1200 nm. In some embodiments, the first optical emitter can be configured to emit light at a first wavelength of from 800 nm to 1000 nm. In various embodiments, the first optical emitter can be configured to emit light at a first wavelength of from 100 nm to 300 nm. In some embodiments, the first optical emitter can be configured to emit light at a first wavelength of from 150 nm to 250 nm. In various embodiments, the first optical emitter 606 can be configured to emit light at a first wavelength that can be greater than or equal to 100 nm, 200 nm, 300 nm, 400 nm, 500 nm, 600 nm, 700 nm, 800 nm, 900 nm, 1000 nm, 1100 nm, 1200 nm, 1300 nm, 1400 nm, 1500 nm, 1600 nm, 1700 nm, 1800 nm, 1900 nm, or 2000 nm, or can be any wavelength falling within a range between any of the foregoing.

The first optical detector 608 can be configured to detect incident light originating from one or more optical emitters, such as the first optical emitter 606. The first optical detector 608 can be configured to detect light after it has propagated into the tissue of a patient to a given depth from the first optical emitter 606 and back to the first optical detector 608. For example, the optical cardiogenic modulation monitoring device 102 can be configured to provide for the propagation of the emitted light through a lung tissue such that propagation of the emitted light occurs from about 1 centimeter (cm) in depth to 5 cm in depth as measured from a surface of the optical cardiogenic modulation monitoring device 102 to a target tissue. In some embodiments, the depth or propagation of the emitter light can be greater than or equal to 1 cm, 2 cm, 3 cm, 4 cm, or 5 cm, or can be an amount falling within a range between any of the foregoing. In various embodiments, the configuration of the optical cardiogenic modulation monitoring device 102 can allow for the propagation of the emitted light through a lung tissue when a surface of the optical cardiogenic modulation monitoring device 102 is directed to the interior of a patient's body toward the surface of the lung tissue and/or the airway. The detected incident light can be used to measure cardiogenic oscillations in lung and other tissue within a patient, as will be discussed elsewhere herein.

It will be appreciated that first optical emitter 606 and first optical detector 608 can be positioned at any location along optical cardiogenic modulation monitoring device 102 to achieve optimal propagation of light into the tissue of a patient. The first optical emitter 606 and first optical detector 608 can be spaced along a length of the optical cardiogenic modulation monitoring device 102 at a distance 610 from 1 cm to 10 cm apart. In some embodiments, a distance 610 between the first optical emitter and the first optical detector can be greater than or equal to 1 cm, 2 cm, 3 cm, 4 cm, 5 cm, 6 cm, 7 cm, 8 cm, 9 cm, 10 cm, 11 cm, 12 cm, 13 cm, 14 cm, or 15 cm, or can be an amount falling within a range between any of the foregoing. In various embodiments, the first optical emitter and the first optical detector are spaced along a planar surface of the optical cardiogenic modulation monitoring device of from 1 cm to 10 cm apart. In various embodiments, the first optical emitter and the first optical detector are spaced along a flat, planar surface of the optical cardiogenic modulation monitoring device of from 1 cm to 10 cm apart. Subject to other factors, the greater distance 610 is, the greater the depth of propagation of light through the tissue.

The optical cardiogenic modulation monitoring device 102 can take on various dimensions in the length, width, and thickness directions. In a particular embodiment herein, it can be approximately 5 to 10 cm in length, 1 cm to 1.5 cm wide, and 0.25 cm to 1.0 cm thick. In some embodiments, the length of optical cardiogenic modulation monitoring device 102 can be greater than or equal to 1 cm, 2 cm, 3 cm, 4 cm, 5 cm, 6 cm, 7 cm, 8 cm, 9 cm, 10 cm, 11 cm, 12 cm, 13 cm, 14 cm, or 15 cm, or can be an amount falling within a range between any of the foregoing. In some embodiments, the optical cardiogenic modulation monitoring device 102 can be about 0.25 cm, 0.5 cm, 0.75 cm, 1.0 cm, or 2.0 cm in width. In some embodiments the width can be in a range wherein any of the foregoing widths can serve as the upper or lower bound of the range, provided that the upper bound is greater than the lower bound. In some embodiments, the optical cardiogenic modulation monitoring device 102 can be about 0.25 cm, 0.50 cm, 0.75 cm or 1.0 cm, 1.25 cm, 1.50 cm, 1.75 cm, 2.0 cm, 2.25 cm, 2.50 cm, or 3.0 cm thick, or can be an amount falling within a range between any of the foregoing. In some embodiments the thickness can be in a range wherein any of the foregoing thicknesses can serve as the upper or lower bound of the range, provided that the upper bound is greater than the lower bound.

In various embodiments, the optical cardiogenic modulation monitoring devices herein can include an optical barrier device disposed between the optical emitters and optical detectors to prevent a direct path for light emitted from an optical emitter to an optical detector. Referring now to FIG. 7, an optical cardiogenic modulation monitoring device 102 is shown in accordance with the embodiments herein. The optical cardiogenic modulation monitoring device 102 can include the features of the optical cardiogenic modulation monitoring device 102 of FIG. 6 and can further include an optical barrier device 702 disposed in between the first optical emitter 606 and the first optical detector 608. In some embodiments, the optical barrier device 702 can be mounted on the surface of the optical cardiogenic modulation monitoring device 102. In some embodiments, the optical barrier device 702 can be at least partially embedded within the surface of the optical cardiogenic modulation monitoring device 102. The optical barrier device 702 can include various materials including light blocking materials such as opaque polymers, metals, dyed materials, and the like.

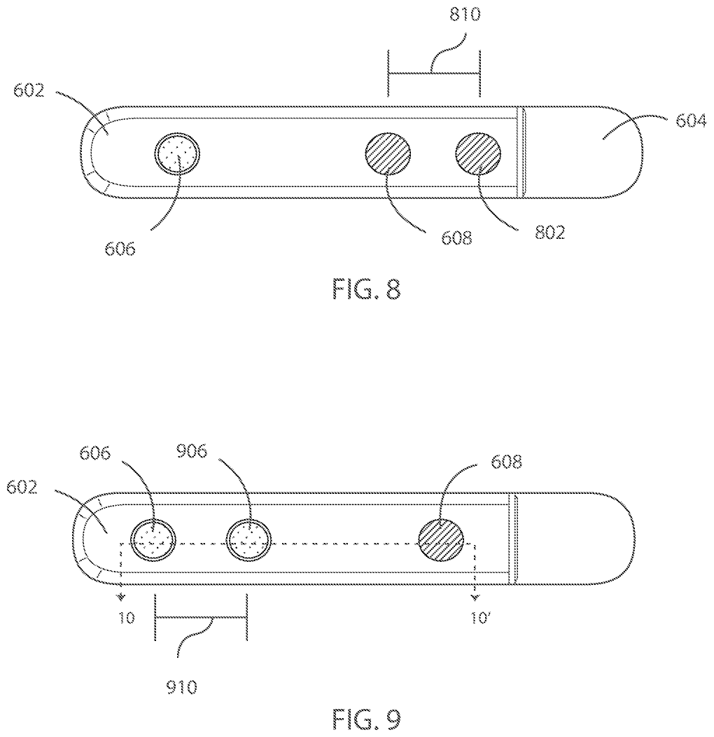

It will be appreciated that the optical cardiogenic modulation monitoring devices embodied herein can include various configurations of multiple optical emitters or optical detectors disposed along a length of the optical cardiogenic modulation monitoring devices. Referring now to FIG. 8, an optical cardiogenic modulation monitoring device 102 is shown in accordance with the embodiments herein. The optical cardiogenic modulation monitoring device 102 can include a housing 602 and a header 604 coupled to the housing 602. The optical cardiogenic modulation monitoring device 102 can include a first optical emitter 606, a first optical detector 608, and a second optical detector 802, each coupled to the housing 602. The first optical emitter 606 and first optical detector 608 can be spaced along a length of the optical cardiogenic modulation monitoring device 102 as discussed in reference to FIG. 6. The first optical detector 608 and the second optical detector 802 can be disposed along a length of the optical cardiogenic modulation monitoring device 102 at a distance 810 of from 1 cm to 5 cm apart. The second optical detector 802 can be configured to detect incident light. In some embodiments, the distance 810 between the first optical detector and the second optical detector can be greater than or equal to 0.25 cm, 0.5 cm, 1 cm, 2 cm, 3 cm, 4 cm, or 5 cm, or more or can be an amount falling within a range between any of the foregoing.

Referring now to FIG. 9, an optical cardiogenic modulation monitoring device 102 is shown in accordance with the embodiments herein. The optical cardiogenic modulation monitoring device 102 can include a housing 602 and a header 604 coupled to the housing 602. The optical cardiogenic modulation monitoring device 102 can include a first optical emitter 606, a second optical emitter 906, and first optical detector 608, each coupled to the housing 602. The first optical emitter 606 and first optical detector 608 can be spaced along a length of the optical cardiogenic modulation monitoring device 102 as discussed in reference to FIG. 6. The first optical emitter 606 and the second optical emitter 906 can be disposed along a length of the optical cardiogenic modulation monitoring device 102 at a distance 910 of from 1 cm to 5 cm or more apart. In some embodiments, the distance 910 between the first optical emitter and the second optical emitter can be greater than or equal to 0.25 cm, 0.5 cm, 1 cm, 2 cm, 3 cm, 4 cm, or 5 cm, or can be an amount falling within a range between any of the foregoing. It will be appreciated, however, that the depth of propagation of light through tissue is dependent on the distance between the emitter and the detector and not the distance between the emitters.

The second optical emitter 906 can be configured to emit light at a second wavelength when present on an optical cardiogenic modulation monitoring device with a first optical emitter. In various embodiments, the second optical emitter 906 can be configured to emit light at a second wavelength different than the first wavelength emitted from the first optical emitter 606. In various embodiments, the second optical emitter 906 can be configured to emit light at a second wavelength of from 150 nm to 2000 nm. In some embodiments, the second optical emitter 906 can be configured to emit light at a second wavelength of from 750 nm to 1500 nm. In various embodiments, the first optical emitter 606 can be configured to emit light at a second wavelength that can be greater than or equal to 800 nm, 900 nm, 1000 nm, 1100 nm, 1200 nm, 1300 nm, 1400 nm, 1500 nm, 1600 nm, 1700 nm, 1800 nm, 1900 nm, or 2000 nm, or can be an amount falling within a range between any of the foregoing. In various embodiments, at least one of the wavelengths of light emitted by the first optical emitter and the second optical emitter is chosen to be more readily transmitted in air than in bodily fluids.

It will be appreciated that optical emitters herein can be used substantially continuously or only during certain time periods. For example, in some cases, the device and/or system can be configured to measure cardiogenic oscillations continuously or substantially continuously. In such a scenario, an optical emitter can be turned on continuously or intermittently as part of a duty cycle such as a certain fraction of time that the emitter is emitting light. It will be appreciated, however, that continuous operation of an emitter may consume substantial energy and lower the battery life of an implanted device. Thus, as a further example, in some embodiments, the device or system can evaluate cardiogenic oscillations only during certain periods of time. For example, the device or system can evaluate cardiogenic oscillations only when it receives a command to measure oxygenation coming from a different device or from a clinician or other system user. As another example, the device or system can evaluate cardiogenic oscillations according to a preset schedule. As another example, the device or system can evaluate cardiogenic oscillations after detecting a particular occurrence or event using one or more sensors, such as an abnormal heart rhythm, an abnormal respiration pattern, or the like. In scenarios where there is more than one emitter, the emitters can be turned on simultaneously or in an alternating pattern.

It will be appreciated that the optical emitters and optical detectors can be formed from various materials. Optical emitters can include a light source such as a light emitting diode (LED), vertical-cavity surface-emitting lasers (VCSELs), electroluminescent (EL) devices, and the like. Optical detectors can include a component selected from the group including of a photodiode, a phototransistor, a charge-coupled device (CCD), a junction field effect transistor (JFET) optical sensor, a complementary metal-oxide semiconductor (CMOS) optical sensor, an integrated photo detector integrated circuit, a light to voltage converter, and the like. Optical emitters and optical detectors are discussed in further detail below.

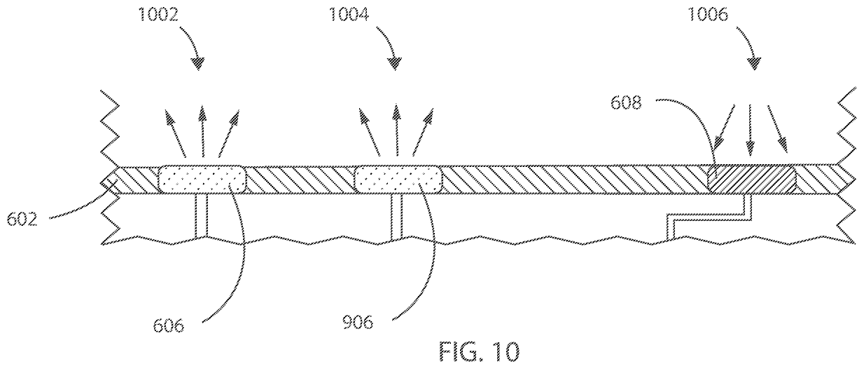

Referring now to FIG. 10, a cross-sectional view of an optical cardiogenic modulation monitoring device is shown in accordance with various embodiments herein. Optical cardiogenic modulation monitoring device 102 includes housing 602 having a first optical emitter 606, a second optical emitter 906, and a first optical detector 608. The first optical emitter 606 can be configured to emit light at a first wavelength 1002 from 100 nm to 2000 nm. The second optical emitter 906 can be configured to emit light at a second wavelength 1004 from 750 nm to 1500 nm. In various embodiments the emitted light of a first wavelength from the first optical emitter can be propagated to a different depth than the emitted light of a second wavelength from the second optical emitter. The first optical detector 608 is configured to detect incident light 1006 that returns to the first optical detector 608 from either the first optical emitter 606, the second optical emitter 906, or both. It will be appreciated that the optical cardiogenic modulation monitoring devices herein can include any combination of one or more optical emitters, optical detectors, and secondary sensors, as described below.

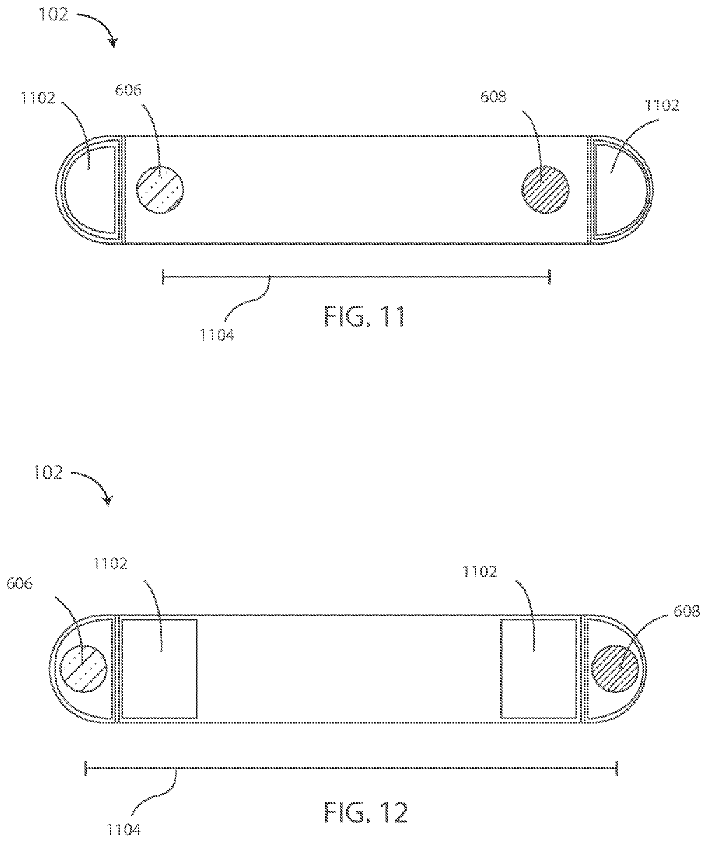

Referring now to FIGS. 11-12, schematic views of additional embodiments of the optical cardiogenic modulation monitoring devices are shown in accordance with various embodiments herein. The optical cardiogenic modulation monitoring devices 102 can include a first optical emitter 606 and a first optical detector 608. While only one optical emitter and one optical detector are shown in the optical cardiogenic modulation monitoring devices 102 of FIGS. 11-12, it will be appreciated that the optical cardiogenic modulation monitoring devices 102 can include more than one optical emitter, more than one optical detector, and a combination of optical emitters and optical detectors. The optical cardiogenic modulation monitoring devices 102 can further include one or more electrodes 1102 disposed along a length of the optical cardiogenic modulation monitoring devices 102. Electrodes 1102 can be configured to function as part of a secondary sensor (such as an ECG sensor) or, in some embodiments, deliver an electrical stimulation a therapy to a patient at or near a treatment site, as will be discussed elsewhere herein.

The first optical emitter 606 and the first optical detector 608, as shown in FIGS. 11 and 12 can be disposed along a length of the optical cardiogenic modulation monitoring device separated by a predetermined distance 1104. Predetermined distance 1104 can include a distance such that the first optical emitter 606 and the first optical detector 608 are spaced along a length of the optical cardiogenic modulation monitoring device from 1 cm to 10 cm apart. In some embodiments, a predetermined distance 1104 between the first optical emitter and the first optical detector can be greater than or equal to 1 cm, 2 cm, 3 cm, 4 cm, 5 cm, 6 cm, 7 cm, 8 cm, 9 cm, 10 cm, 11 cm, 12 cm, 13 cm, 14 cm, or 15 cm, or can be an amount falling within a range between any of the foregoing.

In FIG. 11, the first optical emitter 606 and the first optical detector 608 are positioned between electrodes 1102. In some cases, this can be advantageous because depending on the overall dimensions of the device there may be limited space inside of the device in the areas at the ends of the device. However, in FIG. 12, the first optical emitter 606 and the first optical detector 608 are positioned outside of electrodes 1102. In some cases, this can be advantageous to achieve a maximal spacing between the emitter and detector for a given overall device size. This can be important because a significant factor in the depth of light propagation through the tissue is the distance between the emitter and the detector where a greater distance generally results in a greater depth of propagation through the tissue.

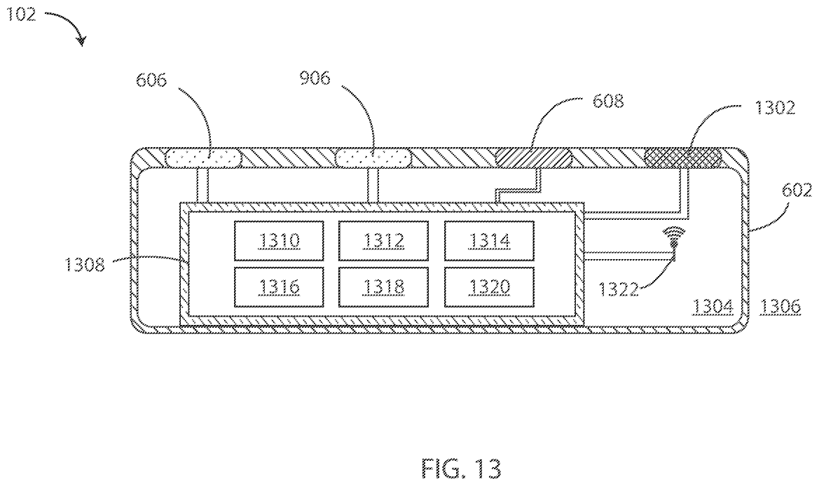

Referring now to FIG. 13, a schematic cross-sectional view of an optical cardiogenic modulation monitoring device 102 is shown in accordance with various embodiments herein. The optical cardiogenic modulation monitoring device 102 can include housing 602. The housing 602 of optical cardiogenic modulation monitoring device 102 can include various materials such as metals, polymers, ceramics, and the like. In some embodiments, all or part of the housing 602 can be transparent and/or can include transparent portions, such as a transparent window. In some embodiments, the housing 602 can be a single integrated unit. In other embodiments, the housing 602 can include housing 602 and header (not shown in this view), as discussed above. In some embodiments, the housing 602, or one or more portions thereof, can be formed of a biocompatible metal, such as titanium. In some embodiments, one or more segments of the housing 602 can be hermetically sealed.

Housing 602 can define an interior volume 1304 that in some embodiments is hermetically sealed off from the area 1306 outside of optical cardiogenic modulation monitoring device 102. The optical cardiogenic modulation monitoring device 102 can include control circuitry 1308. Control circuitry 1308 can include various components, such as components 1310, 1312, 1314, 1316, 1318, and 1320. In some embodiments, some or all of these components can be integrated and in other embodiments these components can be separate. In some embodiments, the components can include one or more of a microprocessor, memory circuitry (such as random-access memory (RAM) and/or read only memory (ROM)), recorder circuitry, telemetry circuitry, measurement circuitry, chemical sensor interface circuitry, power supply circuitry (which can include one or more batteries), normalization circuitry, optical cardiogenic modulation monitoring device control circuitry, optical emitter control circuitry, optical detector control circuitry, and the like. In some embodiments, recorder circuitry can record the data produced by the optical cardiogenic modulation monitoring device and record time stamps regarding the same. In some embodiments, the circuitry can be hardwired to execute various functions, while in other embodiments the circuitry can be implemented as instructions executing on a microprocessor or other computation device. In various embodiments, optical cardiogenic modulation monitoring device further includes a measurement circuit configured to control operation of the optical emitter(s) and the optical detector(s).

A telemetry interface 1322 can be provided for communicating with external devices such as a programmer, a home-based unit, and/or a mobile unit (e.g., a cellular phone, portable computer, etc.) or a wearable medical device. In some embodiments telemetry interface 1322 can be provided for communicating with implanted devices such as a therapy delivery device (e.g., a pacemaker, cardioverter-defibrillator, or the like) or monitoring-only device (e.g., an implantable loop recorder). In some embodiments, the circuitry can be implemented remotely, via either near-field, far-field, conducted, intra-body or extracorporeal communication, from instructions executing on any of the external or the implanted devices, etc. In some embodiments, the telemetry interface 1322 can be located within housing 602. In some embodiments, the telemetry interface 1322 can be located in a header.

Various optical emitters, optical detectors, and other secondary sensors as described elsewhere herein can be in electrical communication with the circuitry. FIG. 13, shows a first optical emitter 606, a second optical emitter 906, a first optical detector 608 and a secondary sensor 1302 in electrical communication with the control circuitry 1308 within the interior volume 1304. In some embodiments, the control circuitry 1308 is configured to selectively activate first optical emitter 606, the second optical emitter 906, the first optical detector 608 and the secondary sensor 1302. It will be appreciated that the control circuitry 1308 can be configured to selectively activate any number of optical emitters, optical detectors, and secondary sensors.

The secondary sensor 1302 can include, but is not limited to, one or more of a pulse oximetry sensor, a chemical sensor, a posture sensor, or a heart rate sensor. The secondary sensor 1302 can be configured to use electrical, optical, pressure, acoustic, or other techniques. In one example, the secondary sensor 1302 can include one or more electrodes to detect electrical properties including, but not limited, to impedance, electrical potential such as in the case of an ECG signal, and the like. In another example, the secondary sensor 1302 can include a microphone or device to detect pressure waves and/or vibration such as an accelerometer to detect characteristics sounds associated with the heart, lungs, or other physiological activity. In another example, the secondary sensor 1302 can include an accelerometer to detect posture. Aspects of exemplary chemical sensors are described in U.S. Pat. Nos. 7,809,441 and 8,126,554, the content of which is herein incorporated by reference.

It will be appreciated that there can be advantages associated with spacing emitters and detectors as far apart as possible to achieve deep tissue propagation of the emitted light. Generally, a limit on maximum spacing is the overall dimensions of the device and/or portions thereof such as the housing. However, in some embodiments, to achieve greater spacing, another structure can be attached to the device and components such as the emitter and/or detector can be mounted thereon. In this way, a maximum distance of separation can be increased based on the length of the structure added. Such a structure can take various forms. In some embodiments, the structure can be substantially rigid while in other embodiments the structure can be flexible.

While not intending to be bound by theory it can be advantageous to include a flexible structure because a long rigid device can be more likely to cause discomfort for the individual into which the device is implanted (in an implantable embodiment). Thus, in various embodiments, the optical cardiogenic modulation monitoring device can include a flexible member. Flexible members embodied herein can assume many shapes, sizes, and configurations suitable for placement within a patient. In some embodiments, the flexible member can be substantially hollow other than components disposed therein. In some embodiments, the flexible member can be non-hollow and filled with a material such as a polymer, a composite, or the like. In contrast, the housing 602 can be substantially rigid. However, in other embodiments the housing 602 itself can also be flexible.

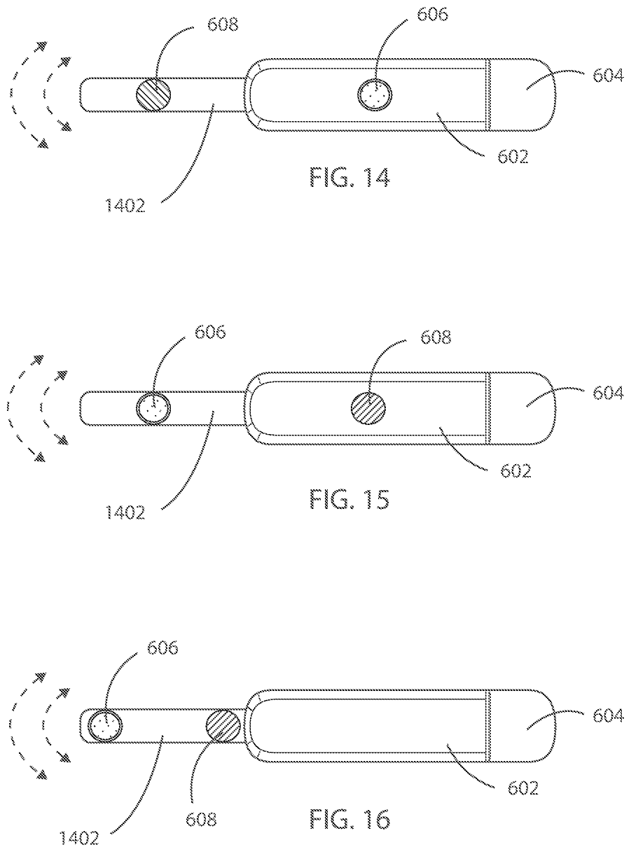

Referring now to FIGS. 14-16 schematic top plan views of optical cardiogenic modulation monitoring devices having flexible members are shown in accordance to various embodiments herein. Specifically referring to FIG. 14, an optical cardiogenic modulation monitoring device 102 is shown including a housing 602 and a header 604 coupled to the housing 602. The optical cardiogenic modulation monitoring device 102 can further include a flexible member 1402 extending form the housing 602. In various embodiments, the flexible member 1402 can be connected to the housing and extend outward in a direction away from the housing 602. As depicted in FIG. 14, the flexible member 1402 can extend outwards from the housing 602 on the opposite side of the housing from the header 604. Alternatively, the flexible member 1402 can extend outwards from the housing 602 on the same side of the housing from the header 604 and/or be an appendage of the header 604 itself. The flexible member 1402 can be constructed form various materials including, for example, a material configured to be bent without damage. In some embodiments, the flexible member 1402 can be formed of a material such as

a polymer, an elastomeric polymer, a flexible composite, or the like. In some embodiments, the flexible member 1402 can be of substantially the same diameter and/or circumference as the housing 602, but in other embodiments can be greater or lesser (such as depicted in FIGS. 14-16) in diameter and/or circumference. While not shown in FIGS. 14-16, one or more conductors can interconnect the flexible member and components disposed thereon such as an emitter and/or detector with the other components of the device that may be housed in housing 602.

It will be appreciated that various combination of optical emitters and optical detectors can be used when a flexible member is present in the optical cardiogenic modulation monitoring devices herein. In various embodiments, the first optical emitter is disposed along a length of the flexible member, while the first optical detector is disposed along the housing. In various embodiments, the first optical detector is disposed along a length of the flexible member, while the first optical emitter is disposed along the housing. In other embodiments, the first optical emitter and the first optical detector are both disposed along a length of the flexible member. However, it is to be understood that various combinations of one or more optical detectors, one or more optical emitters, one or more secondary sensors can be disposed along an optical cardiogenic modulation monitoring device having a flexible member. It will be further understood that in various embodiments, the optical emitters and optical detectors can be disposed together on the same side of the optical cardiogenic modulation monitoring device, while the secondary sensors can be disposed on the opposite side of the optical cardiogenic modulation monitoring device.

The flexible member can take on various dimensions in the length, width, and thickness directions. In a particular embodiment herein, the flexible member can be approximately 1 to 5 cm in length, 0.25 cm to 1.5 cm wide, and 0.25 cm to 1.0 cm thick. In various embodiments, the flexible member is from 1 centimeter (cm) to 3 cm in length. In some embodiments, the length of flexible member 1402 can be greater than or equal to 1 cm, 2 cm, 3 cm, 4 cm, or 5 cm or more, or can be an amount falling within a range between any of the foregoing. In some embodiments, the flexible member 1402 can be about 0.25 cm, 0.5 cm, 0.75 cm, 1.0 cm, or 2.0 cm in width. In some embodiments the length can be in a range wherein any of the foregoing widths can serve as the upper or lower bound of the range, provided that the upper bound is greater than the lower bound. In some embodiments, the flexible member 1402 can be about 0.25 cm, 0.50 cm, 0.75 cm or 1.0 cm, 1.25 cm, 1.50 cm, 1.75 cm, or 2.0 cm thick, or can be an amount falling within a range between any of the foregoing. In some embodiments the thickness can be in a range wherein any of the foregoing thicknesses can serve as the upper or lower bound of the range, provided that the upper bound is greater than the lower bound.

In the embodiment of FIG. 14, the optical cardiogenic modulation monitoring device 102 can include a first optical emitter 606 coupled to the housing and a first optical detector 608 coupled to the flexible member 1402. However, it will be appreciated that the first optical emitter 606 and the first optical detector 608 can be positioned at any location along the optical cardiogenic modulation monitoring device 102, including the flexible member 1402, to achieve optimal propagation of light through the tissue of a patient.

Referring now to FIG. 15, an optical cardiogenic modulation monitoring device 102 is shown in accordance with the embodiments herein. The optical cardiogenic modulation monitoring device 102 can include a housing 602, a header 604 coupled to the housing 602, and a flexible member 1402 extending from the housing 602. In the embodiment of FIG. 15, the optical cardiogenic modulation monitoring device 102 can include a first optical emitter 606 coupled to the flexible member 1402 and a first optical detector 608, coupled to the housing 602.

In some cases, both an emitter and a detector can be disposed on the flexible member. Referring now to FIG. 16, an optical cardiogenic modulation monitoring device 102 is shown in accordance with the embodiments herein. The optical cardiogenic modulation monitoring device 102 can include a housing 602, a header 604 coupled to the housing 602, and a flexible member 1402 extending from the housing 602. The optical cardiogenic modulation monitoring device 102 can include a first optical emitter 606 and a first optical detector 608 each coupled to the flexible member 1402.

While only one optical emitter and one optical detector are shown in the optical cardiogenic modulation monitoring devices 102 of FIGS. 14-16, it will be appreciated that the optical cardiogenic modulation monitoring devices 102 can include more than one optical emitter, more than one optical detector, and a combination of optical emitters and optical detectors disposed anywhere across the length of the optical cardiogenic modulation monitoring device.

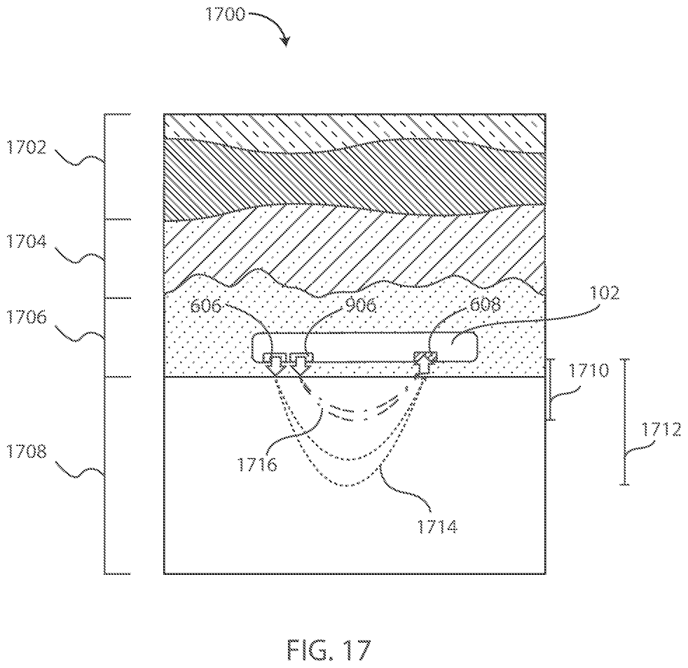

The optical cardiogenic modulation monitoring devices herein can be implanted within various subcutaneous implantation sites or other regions of the inside of the body. Referring now to FIG. 17, a schematic cross-sectional view of a subcutaneous implantation site 1700 with an implanted optical cardiogenic modulation monitoring device 102 is shown in accordance with various embodiments herein. Human skin includes multiple layers including the epidermis 1702 and the dermis 1704. Beneath the layers of human skin and typically above a layer of muscle 1708 is the subcutaneous space that can include a layer of adipose tissue 1706. It will be appreciated that the subcutaneous space further includes additional anatomical structures such as blood vessels, fascia, lymphatic vessels, nervous tissue, hair follicles, and the like.

The optical cardiogenic modulation monitoring device 102 can be disposed at any location within the subcutaneous space. In various embodiments, the optical cardiogenic modulation monitoring device 102 can even be implanted deeper within the body, such as within underlying muscle, within a cavity, such as the chest cavity, adjacent to or within an internal organ of the body, within a blood vessel, or the like. In various embodiments, the optical cardiogenic modulation monitoring device 102 can be held in place via sutures. In some embodiments, the cardiogenic modulation monitoring device 102 can include one or more apertures to facilitate its fixation via sutures. The optical cardiogenic modulation monitoring device 102 can be implanted within a patient such that each of the optical emitters and optical detectors included on the optical cardiogenic modulation monitoring device 102 are disposed facing the interior of a patient's body and are directed toward the volume of the tissue to be monitored. In some embodiments, the tissue to be monitored includes the lung tissue, airway tissue cardiovascular tissue, and the like. Implanting the optical cardiogenic modulation monitoring device 102 facing the interior of a patient's body can selectively direct emitted light toward tissues to be illuminated inside the patient's body.

The optical cardiogenic modulation monitoring device 102 shown in FIG. 17 includes a first optical emitter 606, a second optical emitter 906, and a first optical detector 608. In various embodiments, the optical cardiogenic modulation monitoring device 102 can be configured such that the emitted light 1714 from the first optical emitter 606 at a first wavelength is propagated to a first depth 1710 from a surface of the optical cardiogenic modulation monitoring device 102, and the emitted light 1716 from the second optical emitter 906 at a second wavelength is propagated to a second depth 1712 from a surface of the optical cardiogenic modulation monitoring device 102. In various embodiments, the optical cardiogenic modulation monitoring device 102 can be configured such that emitted light 1714 from the first optical emitter 606 is propagated to a different depth than emitted light 1716 from the second optical emitter 906.

In various embodiments, the depth of emitted light propagated into the tissue of the patient can be tailored by changing the distance between the first optical emitter 606 and second optical emitter 906, and the first optical detector 608, where various distances suitable for use between the optical emitters and optical detectors are discussed elsewhere herein. In some embodiments, the depth of emitted light propagated into the tissue of the patient can also be tailored by selecting various wavelengths of light to be emitted by the first optical emitter 606 and second optical emitter 906. In some embodiments, the first wavelength and second wavelength are different wavelengths. Wavelengths suitable for use in the optical cardiogenic modulation monitoring devices described are discussed elsewhere herein.

Without being bound by any particular theory, it is believed that light generally follows a direct path through a medium in the absence of scattering effects. However, in more complex media where scattering is prevalent, it is believed that light propagation through such media occurs by way of random scattering. Biological tissue is complex, as it is comprised of many tissue types with complex molecules and compounds distributed therein. It is believed that light propagation through biological tissue is due to scattering as caused by the difference in the index of refraction of the various molecules and compounds in the tissues. By way of example, scattering of light can occur in the blood due to the difference of the index of refraction between red blood cells and plasma, and can occur in other tissues due to the difference of the index of refraction between cells, including cellular organelles, and cellular fluids, such as intracellular fluids and extracellular fluids. The resultant path for the emitted light from the optical emitter to the optical detector through biological tissues can assume a broad, arc-shaped optical path as it returns to the optical detector. The arc-shaped optical path becomes deeper as the distance between the emitter and detector increases (all other factors being equal).

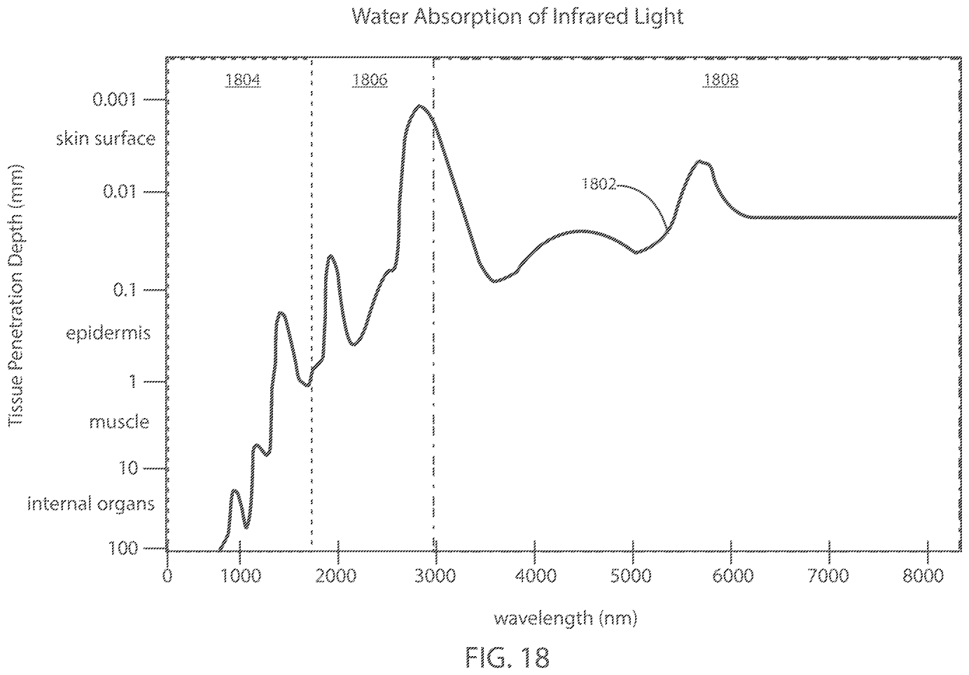

Referring now to FIG. 18, a graphical representation of water absorption of light at various wavelengths illustrating tissue penetration depth is shown in accordance with the embodiments herein. The tissue penetration depth in millimeters (mm) as a function of wavelength in nanometers (nm) is represented as plot 1802. At wavelengths of from 700 nm to 1500 nm, represented in section 1804 of plot 1802, the tissue penetration depth is from roughly 5 mm to 100 mm, which includes depths that penetrate within the epidermis, muscle, internal organs. At wavelengths of from 1500 nm to 3000 nm, represented in section 1806 of plot 1802, the tissue penetration depth is only from roughly 0.1 mm to 0.001 mm, which includes portions of the epidermis and the skin surface. At wavelengths greater than 3000 nm, represented in section 1808 of plot 1802, the tissue penetration depth is from 0.1 mm or less, which includes superficial portions of the epidermis and the skin surface. Thus, as can be seen, the specific wavelength chosen impacts the maximum depth of propagation through the tissue. In embodiments herein, wavelengths are selected (as also described elsewhere herein) to provide for deep tissue propagation of the emitted light.

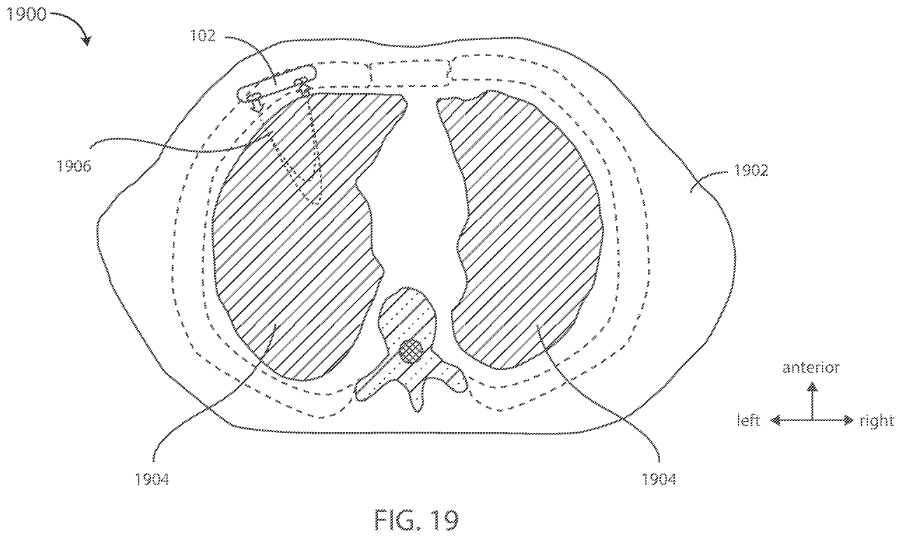

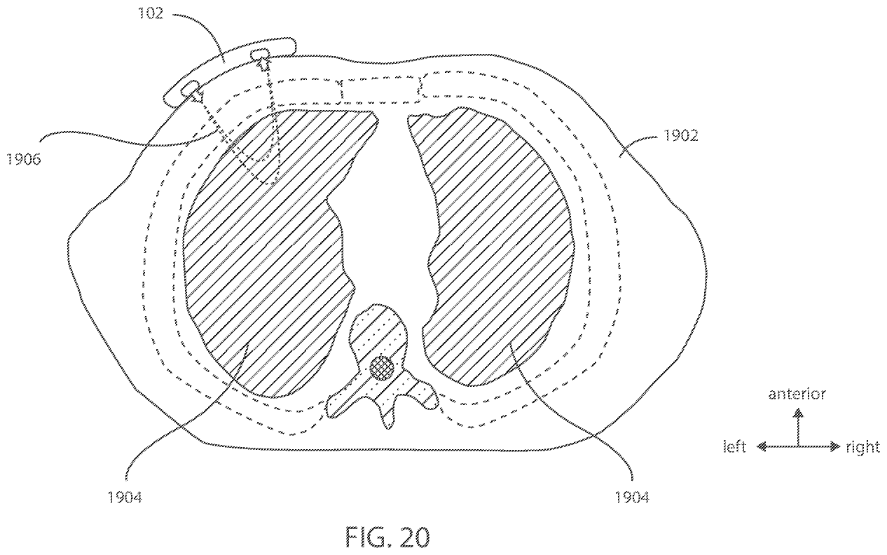

Referring now to FIG. 19, a schematic cross-sectional diagram of a human thorax 1902 with an implanted optical cardiogenic modulation monitoring device positioned therein is shown in accordance with the embodiments herein. The cross-sectional view 1900 of the human thorax 1902 includes the left and right lungs 1904. An optical cardiogenic modulation monitoring device 102 is shown positioned within an intercostal space with an optical emitter and an optical detector disposed facing a surface of the lung tissue. The optical cardiogenic modulation monitoring device 102 is shown disposed facing the interior of a patient's body and is directed toward the surface of the tissue to be monitored. In some embodiments, the tissue to be monitored includes the airway, lung tissue, cardiovascular tissue, and the like. Implanting the optical cardiogenic modulation monitoring device 102 facing the interior of a patient's body can selectively direct emitted light 1906 toward tissues to be illuminated inside the patient's body.

Referring now to FIG. 20, a schematic cross-sectional diagram of a human thorax 1902 with a wearable optical cardiogenic modulation monitoring device positioned thereon is shown in accordance with the embodiments herein. The cross-sectional view 1900 of the human thorax 1902 includes the left and right lungs 1904. An optical cardiogenic modulation monitoring device 102 having an optical emitter and optical detector is shown externally positioned on the skin over an intercostal space. The optical cardiogenic modulation monitoring device 102 can be held in place using a wearable strap or another device. The optical cardiogenic modulation monitoring device 102 is shown disposed facing the interior of a patient's body and is directed toward the surface of the tissue to be monitored. In some embodiments, the tissue to be monitored includes the airway tissue, lung tissue, cardiovascular tissue, or any other anatomical structure of interest in the body, and the like. Implanting the optical cardiogenic modulation monitoring device 102 with the emitter(s) and detector(s) thereof facing the interior of a patient's body can selectively direct emitted light 1906 toward tissues to be illuminated inside the patient's body.

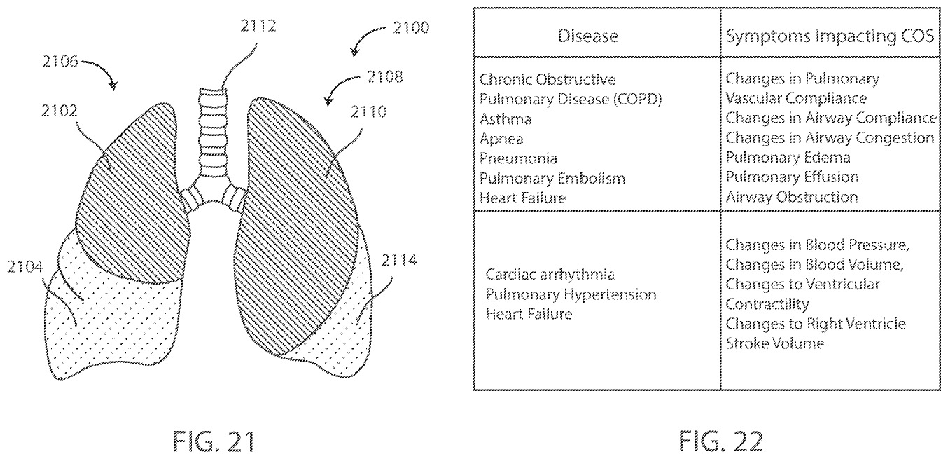

Referring now to FIG. 21, a schematic view of human lungs is shown in accordance with various embodiments herein. The human pulmonary system 2100 includes the right lung 2106, the left lung 2108, and the trachea 2112 and associated bronchial tree. Right lung 2106 includes a right upper portion 2102 and a right lower portion 2104. Left lung 2108 includes a left upper portion 2110 and a left lower portion 2114. Such tissues can be evaluated using embodiments of optical cardiogenic modulation monitoring devices herein to detect cardiogenic oscillations.

Cardiogenic oscillations can be impacted by a number of factors. In healthy patients, cardiogenic oscillations are impacted by factors including but not limited to respiratory phase, activity level, and posture, and stress level. Cardiogenic oscillations can also be affected by a number of disease states.

Referring now to FIG. 22, a table detailing disease sates and their symptoms that affect cardiogenic oscillations is shown in accordance with the embodiments herein. In various embodiments, cardiogenic oscillations are affected by lung and airway conditions that affect pulmonary vascular compliance, airway compliance, airway congestion, pulmonary edema, pulmonary effusion, airway obstruction. Examples of such conditions can include COPD, Asthma, Apnea, Pneumonia, Pulmonary embolism, and Heart Failure. Cardiogenic oscillations can also be impacted by heart conditions that affect Pulmonary artery blood pressure, Pulmonary artery blood volume, right ventricular contractility, and right ventricle stroke volume. Examples of such conditions include cardiac arrhythmia, pulmonary hypertension, and heart failure.

### Cardiogenic Oscillation Characteristics

Cardiogenic oscillations are heart-synchronous variations in the gas, flow, and pressure of the airway. The morphology of cardiogenic oscillations is not completely understood but it is widely accepted that respiratory airflow and pressure are affected by cardiac contractions. In particular, cardiogenic oscillations are caused by blood flow and pressure changes due to the right ventricular contraction of the heart.

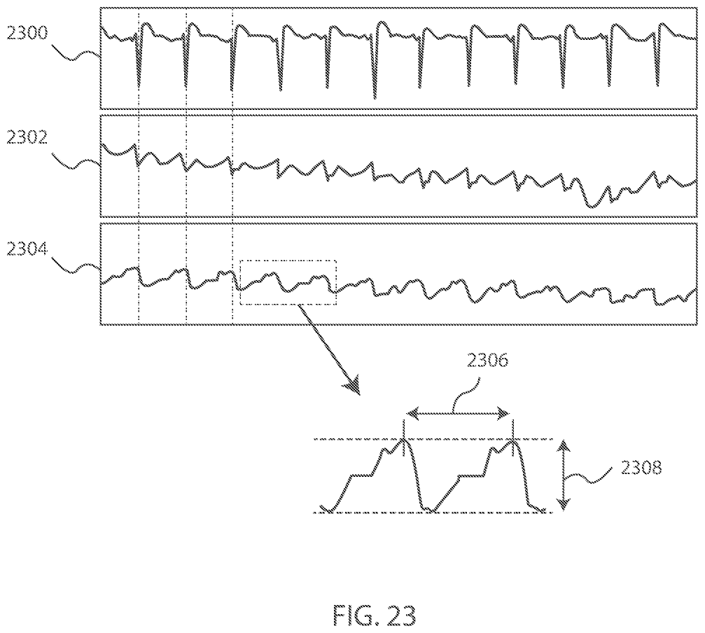

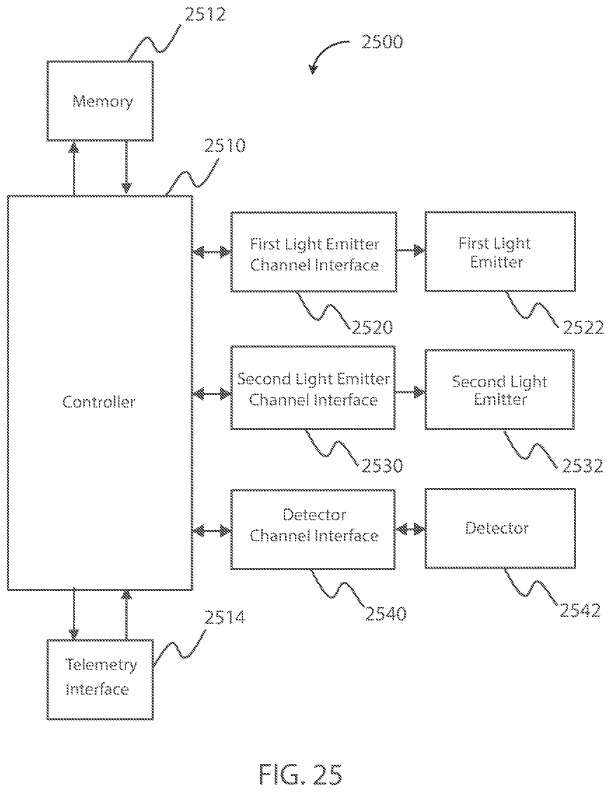

Referring now to FIG. 23 a graphical representation depicting the magnitude and morphology cardiogenic oscillations is shown in accordance with various embodiments herein. FIG. 23 depicts an electrocardiogram (ECG) signal 2300, a cardiogenic oscillation airway pressure signal (COS_{paw}) 2302, and a cardiogenic oscillation airway flow signal (COS_{flow}) 2304. The ECG signal is generated from the electrical activity of the heart. The ECG signal is approximately periodic with each period corresponding to a cardiac cycle. The vertical dashed lines denote each period of the ECG signal. FIG. 23 shows that COS_{paw} and COS_{flow} 2302, 2304 are also approximately periodic and have the same period as the ECG signal. The bottom of FIG. 25 shows an enlarged version of COS_{flow} having period 2306 and amplitude 2308. Amplitude 2308 can reflect the relative magnitude of cardiogenic oscillations. Similarly, the amplitude of the waveform in the cardiogenic oscillation airway pressure signal (COS_{paw}) 2302 can reflect the relative magnitude of cardiogenic oscillations. The period 2306 is approximately the same as the period of the ECG signal 2300 and the COS_{flow} 2304. Based on changes to the optical path of light travelling through lung tissue and/or airway tissue associated with fluctuations in airway pressure and airway flow induced by cardiogenic oscillations, the relative magnitude of cardiogenic oscillations, as well as the waveform thereof, can be derived based on the optical properties of light as it returns to optical cardiogenic modulation monitoring device after propagation through the tissue.

### Cardiogenic Oscillation Measurements

In various embodiments, the optical cardiogenic modulation monitoring device is configured to determine at least one of a magnitude and a morphology of cardiogenic oscillations. In various embodiments, at least one ratiometric property of the incident light measured by the optical detector is used to assess cardiogenic oscillations of the lung tissue including, but not limited to absorption, scattering, and phase. In various embodiments, the absorption measured by the optical detector is used to determine an optical density of an optical path from the optical emitter to the optical detector and the optical density is used to assess the cardiogenic oscillations. Without wishing to be bound by any particular theory, it is believed that a tissue with a higher optical density at a certain wavelength will absorb more of the light along the optical path from the optical emitter to the optical detector, resulting in a lower level of light incident on the optical detector at that wavelength.

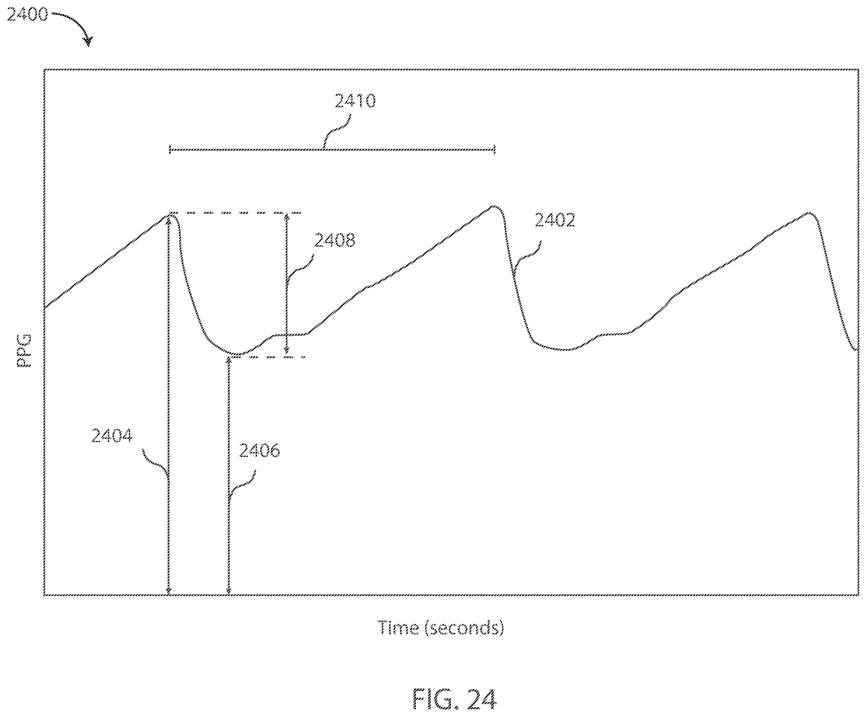

In various embodiments the optical path from the emitter to the detector will contain both tissue and air. In some conditions, the optical density of the tissue experiences little to no change during the course of a cardiogenic oscillation. However, the ratio of tissue-to-air along the optical path and/or the optical path length can still change during a cardiogenic oscillation. These cyclical changes arise from the fluctuations in airway pressure and air flow due to cardiogenic oscillations as shown in FIG. 24. The changes in the ratio of tissue-to-air along the optical path and/or the optical path length will affect the optical density impacting light propagating through the optical path. In some embodiments, the magnitude and fluctuation of the optical density as detected by light returning to the optical detector can be indicative of the magnitude and morphology of cardiogenic oscillations in a patient. In some embodiments, the optical emitter can emit light at a wavelength selected to be more readily absorbed by body fluids than by air (or vis versa), such that modulations in the airway, pressure and flow will affect the optical path of the emitted light and optical density of the light received by the detector.

In various embodiments, the optical cardiogenic modulation monitoring device is configured to determine a trend, average, or statistical measure of at least one of a magnitude and a morphology of the cardiogenic oscillations over a time period. In some embodiments, the time period can be greater than or equal to 1 hour, 1 day, 2 days, one week, one month, one or can be an amount falling within a range between any of the foregoing. In some embodiments, the time period can be less than or equal to 1 day, 1 hour, 1 minute, or can be an amount falling within a range between any of the foregoing.

In various embodiments, the optical cardiogenic modulation monitoring device is configured to determine respiratory system mechanics based on the cardiogenic oscillations. The optical cardiogenic modulation monitoring device can detect decreasing magnitude of cardiogenic oscillations over time, which can reflect decreasing compliance of a respiratory system of a patient. Conversely, in some embodiments the optical cardiogenic modulation monitoring device can detect increasing magnitude of cardiogenic oscillations over time, which can reflect increasing compliance of a respiratory system of a patient. In various embodiments, the optical cardiogenic monitoring device is further configured to determine one of a heart rate, a respiratory rate, a tidal volume, or an extravascular lung water concentration.

Many factors can influence the value of the measurement (e.g., tissue type, emitter energy, detector sensitivity) by the optical detector. Consequentially, a ratiometric calculation can be applied to obtain an assessment of the cardiogenic oscillations. In various embodiments the cardiogenic oscillations of the lung tissue are measured based on a ratiometric calculation including the incident light on the detector originating from the light emitted at the first wavelength and the incident light on the detector originating from the light emitted at the second wavelength. Cardiogenic oscillations can depend on a ratio of energies (E₁/E₂) detected by an optical detector of an optical cardiogenic modulation monitoring device.

$$cardiogenic oscillations ∝ \frac{{E}_{1}}{{E}_{2}}$$

The ratio of energies (E₁/E₂) can be a ratio of the energy detected by the optical detector at a first wavelength (E₁) to the energy detected by the optical detector at a second wavelength (E₂). Alternatively, the ratio of energies (E₁/E₂) can be a ratio of the energy detected by the optical detector traveling from a first emitter at a first distance from the optical detector (E₁) to the energy detected by the optical detector traveling from a first emitter at a first distance from the optical detector (E₂).

As previously discussed, the optical cardiogenic modulation monitoring device can further include one or more secondary sensors (e.g., pulse oximetry sensor, chemical sensor, posture sensor, and heart rate sensor). In various embodiments, the cardiogenic oscillations can be interpreted in view of at least one of the secondary signals (e.g., heart rate, respiration, circadian rhythm, and posture).

### Emitter/Detector Characteristics

In some embodiments, the one or more optical emitters can include solid state light sources such as GaAs, GaAlAs, GaAlAsP, GaAlP, GaAsP, GaP, GaN, InGaAlP, InGaN, ZnSe, or SiC light emitting diodes or laser diodes that excite the sensing one or more optical detectors at or near the wavelength of maximum absorption for a time sufficient to emit a return signal. However, it will be understood that in some embodiments the wavelength of maximum absorption/reflection varies as a function of the optical path from the one or more optical emitters to the one or more optical detectors.

In some embodiments, the one or more optical emitters can include other light emitting components including incandescent components. In some embodiments, the optical emitters can include a waveguide. The optical emitters can also include one or more filters such as bandpass filters, high pass filter, low pass filters, and/or other components such as antireflection elements, and/or focusing optics.

In some embodiments, the one or more optical emitters can include a plurality of LEDs with bandpass filters, with each of the LED-filter combinations emitting at a different center frequency. According to various embodiments, the LEDs can operate at different center-frequencies, sequentially turning on and off during a measurement. As multiple different center-frequency measurements are made sequentially, a single unfiltered optical detector can be used in some embodiments. However, in some embodiments, a polychromatic source can be used with multiple detectors that are each bandpass filtered to a particular center frequency.

The one or more optical detectors can be configured to receive light from the optical emitters. In an embodiment, the optical detectors can include a component to receive light. By way of example, in some embodiments, the optical detectors can include a charge-coupled device (CCD). In other embodiments, the optical detectors can include a photodiode, a junction field effect transistor (JFET) type optical sensor, or a complementary metal-oxide semiconductor (CMOS) type optical sensor. In some embodiments, the optical detectors can include an array of optical detecting components. In some embodiments, the optical detectors can include a waveguide. The one or more optical detectors can also include one or more bandpass filters and/or focusing optics. In some embodiments, the optical detectors can include one or more photodiode detectors, each with an optical bandpass filter tuned to a specific wavelength range.

Referring now to FIG. 25, a schematic block diagram of some components 2500 of an optical cardiogenic modulation monitoring device is shown in accordance with various embodiments herein. It will be appreciated that some embodiments can include additional elements beyond those shown in FIG. 25. In addition, some embodiments may lack some elements shown in FIG. 25. The implantable sensing devices herein can gather information through one or more detecting channels. A controller 2510 can communicate with a memory 2512 via a bidirectional data bus. It will be appreciated that controller 2510 can include one or more microprocessors. The memory 2512 can include read-only memory (ROM) or random-access memory (RAM) for program storage and RAM for data storage, or any combination thereof. The implantable medical device can include one or more optical emitters 2522, 2532, one or more detectors 2542, or one or more other sensors (not pictured). The one or more other sensors, but are not to be limited to, a pulse oximetry sensor, a chemical sensor, a posture sensor, or a heart rate sensor. The optical cardiogenic modulation monitoring device can further include a measurement circuit configured to control operation of the optical emitters and the optical detectors.

Each optical emitter 2522, 2532 is communicatively coupled to an optical emitter channel interface 2520, 2530. Each detector 2542 is communicatively coupled to a detector channel interface 2540. Each other sensor is communicatively coupled to a separate and other sensor channel interface (not pictured). Each of the optical emitter channel interfaces 2520, 2530, the detector channel interface 2540, and any of the other sensor channel interfaces can communicate with controller 2510.

The first optical emitter channel interface 2520, the second optical emitter channel interface 2530, and the detector channel interface 2540 can each include various components such as analog-to-digital converters for digitizing signal inputs, sensing amplifiers, registers which can be written to by the control circuitry in order to adjust the gain and threshold values for the sensing amplifiers, source drivers, modulators, demodulators, multiplexers, and the like. A telemetry interface 2514 is also provided for communicating with external devices such as a programmer, a home-based unit, and/or a mobile unit (e.g., a cellular phone, portable computer, etc.), implanted devices such as a pacemaker, cardioverter-defibrillator, loop recorder, and the like.

### Tissue Penetration

Spacing of the optical emitters and optical detectors along a length of the optical cardiogenic modulation monitoring devices herein can determine the depth of penetration of the emitted light into the surrounding tissues. By way of non-limiting example, the penetration depth of emitted light can be approximated according to the following equation:

light penetration depth=˜½(spacing between emitter and detector).

In some embodiments, the depth or propagation of light from the emitter light can be greater than or equal to 1 cm, 2 cm, 3 cm, 4 cm, or 5 cm, or can be an amount falling within a range between any of the foregoing. In various embodiments, the configuration of the optical cardiogenic modulation monitoring device can allow for the propagation of the emitted light into a patient's body and into the lung tissue. In some embodiments, the length between the first optical emitter and the first optical detector can be greater than or equal to 1 cm, 2 cm, 3 cm, 4 cm, 5 cm, 6 cm, 7 cm, 8 cm, 9 cm, 10 cm, 11 cm, 12 cm, 13 cm, 14 cm, or 15 cm, or can be an amount falling within a range between any of the foregoing. In various embodiments, at least 50, 60, 70, 80, 90, 95, 98, or 99 percent of the light incident upon the optical detector and used to measure cardiogenic oscillations of the lung tissue has propagated through a tissue at a depth of at least 1 cm, 2 cm, 3 cm, 4 cm, or 5 cm or more as measured from a surface of the device and/or the optical emitter(s) thereof.

### Therapies

The devices and systems herein can be configured to suggest, initiate and/or deliver a therapy to a patient in response to a change in cardiogenic oscillations detected by the optical cardiogenic modulation monitoring devices herein. In various embodiments, the optical cardiogenic modulation monitoring devices can be configured to deliver a therapy to a patient. In some embodiments, implantable therapeutic devices can be implanted and used in a system in conjunction with the wearable optical cardiogenic modulation monitoring devices described herein. The implantable therapeutic devices can include, but not be limited to cardiac rhythm management devices, an implantable cardioverter-defibrillator (ICD), a left ventricular assist device (LVAD), a pacemaker, and the like.

In various embodiments, the devices and systems herein can be configured to deliver or modify a pulmonary oxygenation therapy to a patient to treat one or more diseases states or conditions including, but not limited to, chronic obstructive pulmonary disease (COPD), pulmonary edema with heart failure with normal cardiac output or reduced cardiac output, asthma, pneumonia, various airway obstructions, and pulmonary embolism. In some embodiments, a pulmonary oxygenation therapy can include a command sent to a device such as a ventilation device or other breathing assistance device to change proportions of gases being provided (such as increasing or decreasing oxygen content), change volumes of gases being provided, or the like.

### AC/DC Component Calculations

Referring now to FIG. 24, a graphical representation an optical signal is shown in accordance with the embodiments herein. The optical signal 2400 can be indicative of the incident light measured the optical detector of an optical cardiogenic oscillations measurement device. As depicted by FIG. 24, the optical signal can be approximately periodic having period 2410, maximum value 2404, and minimum value 2406. In this example, the periodicity of the signal is a consequence of the systole and diastole of the cardiac cycle. During systole, the heart contracts, pushing the oxygenated blood towards the periphery of the body, resulting in an increased blood pressure. During diastole, the hear fills with blood, retracting the blood from the periphery of the body resulting in a decrease in blood pressure. Due to the absorption of light by various chromophores in blood, such as oxyhemoglobin and deoxyhemoglobin, the optical density of the tissue at certain wavelengths increases during systole and decreases during diastole resulting, in a periodic signal with maximums and minimums. Each cycle of the signal contains a maximum value 2404 at the systolic peak, and minimum value 2406 at the diastolic valley.

The optical signal can effectively have an AC (or variable) component 2408 and a DC (or constant) component 2402. The term ‘AC’, as used herein, refers to a portion of signal that varies relatively rapidly with time. The AC component can be indicative of the portion of the signal originating by pulsations in a patient's blood during each heartbeat. The term ‘DC’, as used herein signals, refers to portions of the signal that are relatively invariant with time.

As shown and discussed in FIG. 24, the incident light measured by the optical detector of a cardiogenic oscillations monitoring device can have both an AC and DC components. In various embodiments, both an AC component and a DC component of the light detected by the optical detector are used to determine a property of the tissue (e.g., cardiogenic oscillations). In various embodiments, the property can be derived from a ratio of the AC and DC components of the signal as measured by the optical detector. In various embodiments, the property can be derived from a ratio of the AC and DC components of the signal measured by the optical detector at two distinct wavelengths. For instance, the property can be derived using a ratio of ratio (RoR) calculation according to the following calculation:

$$cardiogenic oscillations ∝ \frac{A {C}_{1} / D {C}_{1}}{A {C}_{2} / D {C}_{2}}$$

where AC₁ is the AC component of the signal measured by the optical detector at the first wavelength; DC₁ is the DC component of the signal measured by the optical detector at the first wavelength; AC₂ is the AC component of the signal measured by the optical detector at the second wavelength; and DC₂ is the DC component of the signal measured by the optical detector at the second wavelength.

### Methods

Many different methods are contemplated herein, including, but not limited to, methods of making, methods of using, and the like. Aspects of system/device operation described elsewhere herein can be performed as operations of one or more methods in accordance with various embodiments herein.

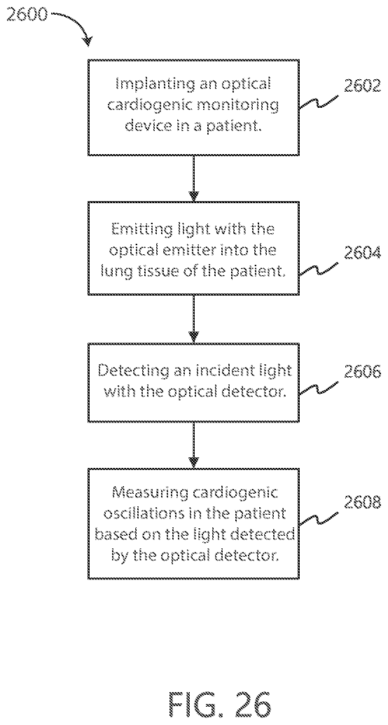

Referring now to FIG. 26, a method 2600 for measuring cardiogenic oscillations in a patient is included. The method can include implanting an optical cardiogenic monitoring device in a patient at 2602 where the optical cardiogenic monitoring device can include an optical emitter and an optical detector. The method can include emitting light with the optical emitter into the lung tissue of the patient at 2604. The method can include detecting incident light with the optical detector at 2606. The method can include measuring cardiogenic oscillations in the patient based on the light detected by the optical detector at 2608.

In an embodiment of the method, implanting the optical cardiogenic monitoring device comprises implanting the optical cardiogenic monitoring device subcutaneously at or near a site of the lung tissue.

In an embodiment of the method, implanting the optical cardiogenic monitoring device subcutaneously comprises implanting the optical cardiogenic monitoring device in an intercostal space at or near the site of the lung tissue.

In an embodiment of the method, the optical cardiogenic monitoring device is implanted at a distance of from 5 mm to 50 mm from a surface of the lung tissue.

In an embodiment of the method, emitting light with the optical emitter into the lung tissue of the patient comprises emitting the light at a wavelength from 800 nm to 1000 nm.

In an embodiment of the method, emitting light with the optical emitter into the lung tissue of the patient comprises emitting the light at least two distinct wavelengths.

In an embodiment of the method, implanting an optical cardiogenic monitoring device in a patient comprises orienting the optical cardiogenic monitoring device such that optical emitter and the optical detector face internally relative to the skin of the patient.

In an embodiment of the method, emitting light with the optical emitter into the lung tissue of the patient comprises propagating the light from 1 cm to 5 cm into the tissue of the patient relative to a surface of the optical cardiogenic monitoring device.

### Systems

The devices herein can be used in various systems including multiple optical cardiogenic modulation monitoring devices and secondary sensors, including, but not limited to, methods of making, methods of using, and the like. Aspects of optical cardiogenic modulation monitoring devices described elsewhere herein can be included of one or more embodiments of a system in accordance with various embodiments herein.

In an embodiment, an optical cardiogenic monitoring system is included for detecting cardiogenic oscillations of a patient. The sensor system can include a housing. The sensor system can include an optical cardiogenic monitoring device. The optical cardiogenic monitoring device can include an optical emission assembly. The optical emission assembly can include a first optical emitter configured to emit light at a first wavelength and a second optical emitter configured to emit light at a second wavelength. The optical cardiogenic monitoring device can include an optical detector configured to detect incident light. In the systems described herein the light emitted from the optical emitter propagates through lung tissue. In the systems described herein, the detected incident light is used to measure cardiogenic oscillations of the lung tissue. In the systems described herein, the optical cardiogenic monitoring device is configured to be implanted in a subcutaneous tissue of a patient.

In an embodiment of the system at least one of absorption, scattering and phase of the light detected by the optical detector is used to measure the cardiogenic oscillations.

In an embodiment of the system, both an AC component and an DC component of the light detected by the optical detector are used to measure the cardiogenic oscillations of the lung tissue.

In an embodiment of the system, the first wavelength is from 800 nm to 1000 nm.

In an embodiment of the system, the first wavelength is from 150 nm to 250 nm.

In an embodiment of the system, the second wavelength is a near-infrared wavelength.

In an embodiment of the system, the second wavelength is from 750 nm to 1500 nm.

In an embodiment of the system, the cardiogenic oscillations of the lung tissue are a function of the ratio of the detected light originating from the first optical emitter and the detected light originating from the second optical emitter.

In an embodiment of the system, the housing comprising a flexible member extending from the housing, wherein at least one of the first optical emitter, the second optical emitter, and the optical detector are disposed on a flexible member.

In an embodiment of the system, the optical emission assembly and the optical detector are disposed on a substantially planar surface.

In an embodiment of the system, the optical emitter is disposed from 1 cm to 10 cm away from the optical detector.

In an embodiment of the system, the optical cardiogenic monitoring device is further configured to determine one of a heart rate, a respiratory rate, a tidal volume, or an extravascular lung water concentration.

In an embodiment of the system, the light from the optical emission assembly propagates a depth of 1 cm to 5 cm into the lung tissue as measured from a surface of the optical cardiogenic monitoring device.

In an embodiment of the system, the system further includes at least one of a pulse oximetry sensor, chemical sensor, posture sensor, and heart rate sensor.

In an embodiment of the system, the cardiogenic oscillations measurements are interpreted in view of at least one of heart rate, respiration, circadian rhythm, and posture.

It should be noted that, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as arranged and configured, constructed and arranged, constructed, manufactured and arranged, and the like.

All publications and patent applications in this specification are indicative of the level of ordinary skill in the art to which this invention pertains. All publications and patent applications are herein incorporated by reference to the same extent as if each individual publication or patent application was specifically and individually indicated by reference.

As used herein, the recitation of numerical ranges by endpoints shall include all numbers subsumed within that range (e.g., 2 to 8 includes 2.1, 2.8, 5.3, 7, etc.).

The headings used herein are provided for consistency with suggestions under 37 CFR 1.77 or otherwise to provide organizational cues. These headings shall not be viewed to limit or characterize the invention(s) set out in any claims that may issue from this disclosure. As an example, although the headings refer to a “Field,” such claims should not be limited by the language chosen under this heading to describe the so-called technical field. Further, a description of a technology in the “Background” is not an admission that technology is prior art to any invention(s) in this disclosure. Neither is the “Summary” to be considered as a characterization of the invention(s) set forth in issued claims.

The embodiments described herein are not intended to be exhaustive or to limit the invention to the precise forms disclosed in the following detailed description. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

The claims are:

1. An optical cardiogenic modulation monitoring device comprising:
an optical emitter, wherein the optical emitter is configured to emit light at a first wavelength; and
an optical detector, wherein the optical detector is configured to detect incident light;
wherein the monitoring device is configured so that light emitted from the optical emitter propagates through lung tissue; and
wherein the monitoring device is configured to use detected incident light to measure cardiogenic oscillations of the lung tissue.

2. The monitoring device of claim 1, wherein the optical cardiogenic monitoring device is configured to be implanted in a subcutaneous tissue of a patient.

3. The monitoring device of claim 1, wherein at least one of absorption, scattering and phase of the light detected by the optical detector is used to measure cardiogenic oscillations of the lung tissue.

4. The monitoring device of claim 3, wherein the absorption is used to determine an optical density of an optical path from the optical emitter to the optical detector and the optical density is used to measure the cardiogenic oscillations of the lung tissue.

5. The monitoring device of claim 1, wherein both an AC component and an DC component of the light detected by the optical detector are used to determine used to measure the cardiogenic oscillations of the lung tissue.

6. The monitoring device of claim 1, wherein the first wavelength is from 800 nm to 1000 nm.

7. The monitoring device of claim 1, wherein the first wavelength is from 150 nm to 250 nm.

8. The monitoring device of claim 1, further comprising a second optical emitter;
wherein the second optical emitter is configured to emit light at a second wavelength.

9. The monitoring device of claim 8, wherein the second wavelength is a near-infrared wavelength.

10. The monitoring device of claim 8, wherein the second wavelength is from 750 nm to 1500 nm and different than the first wavelength.

11. The monitoring device of claim 1, further comprising a housing comprising a flexible member extending from the housing, wherein at least one of the optical emitter and the optical detector are disposed on a flexible member.

12. The monitoring device of claim 1, wherein the optical emitter is disposed from 1 cm to 10 cm away from the optical detector.

13. The monitoring device of claim 1, wherein the light from the optical emitter propagates a depth of 1 cm to 5 cm into the lung tissue as measured from a surface of the optical cardiogenic monitoring device.

14. The monitoring device of claim 1, further comprising at least one of a pulse oximetry sensor, chemical sensor, posture sensor, and heart rate sensor.

15. The monitoring device of claim 14, wherein the cardiogenic oscillation measurements are interpreted in view of at least one of heart rate, respiration, circadian rhythm, and posture.

16. A method for measuring cardiogenic oscillations in a patient comprising:
implanting an optical cardiogenic monitoring device in a patient, the optical cardiogenic monitoring device comprising an optical emitter and an optical detector;
emitting light with the optical emitter into the lung tissue of the patient;
detecting incident light with the optical detector; and
measuring cardiogenic oscillations in the patient based on the light detected by the optical detector.

17. The method of claim 16, wherein implanting the optical cardiogenic monitoring device comprises implanting the optical cardiogenic monitoring device subcutaneously at or near a site of the lung tissue.

18. The method of claim 17, wherein implanting the optical cardiogenic monitoring device subcutaneously comprises implanting the optical cardiogenic monitoring device in an intercostal space at or near the site of the lung tissue.

19. The method of claim 16, wherein emitting light with the optical emitter into the lung tissue of the patient comprises emitting the light at a wavelength from 800 nm to 1000 nm.

20. The method of claim 16, wherein emitting light with the optical emitter into the lung tissue of the patient comprises propagating the light from 1 cm to 5 cm into the tissue of the patient relative to a surface of the optical cardiogenic monitoring device.
